(19) United States
(12) Patent Application Publication
(10) Pub. No.: US 2025/0332368 A1
(43) Pub. Date: Oct. 30, 2025

(54) SYSTEM, DEVICES AND METHODS FOR DELIVERING A FLOW OF OXYGEN

(71) Applicants: CARILION CLINIC, Roanoke, VA (US); VIRGINIA TECH INTELLECTUAL PROPERTIES, INC., Blacksburg, VA (US)

(72) Inventors: Umar F. SOFI, Roanoke, VA (US); Hunter T. MOLLICA, Blacksburg, VA (US); Alfred L. WICKS, Blacksburg, VA (US)

(73) Assignees: CARILION CLINIC, Roanake, VA (US); VIRGINIA TECH INTELLECTUAL PROPERTIES, INC., Blacksburg, VA (US)

(21) Appl. No.: 18/868,393

(22) PCT Filed: May 23, 2023
(86) PCT No.: PCT/US2023/023276

§ 371 Date: Nov. 22, 2024

Related U.S. Application Data
(60) Provisional application No. 63/345,675, filed on May 25, 2022.

(51) Int. Cl. A61M 16/06; A61M 16/00

(52) U.S. Cl. CPC A61M 16/0672; A61M 16/024; A61M 2016/0027; A61M 2016/0039; A61M 2230/20

(57) ABSTRACT

An apparatus includes a nasal cannula having a nasal prong. The nasal prong includes a first end portion that defines an inlet opening, a second end portion that defines an outlet opening, and a middle portion. A side wall of the nasal prong defines a flow passage between the inlet opening and the outlet opening. The nasal prong is configured to be inserted within a nostril of a patient with the outlet opening disposed within the nostril. The inlet opening is fluidically coupled to a support tube to deliver a flow of the gas into the airway of the patient via the nasal prong. The side wall of the nasal prong defines a port that is in fluid communication with the flow passage. The port is fluidically coupled to a pressure sensor to take a series of pressure measurements within the flow passage during the delivery of the oxygen-containing gas.

### CROSS-REFERENCE TO RELATED APPLICATIONS

This application claims priority to and the benefit of U.S. Provisional Patent Application No. 63/345,675, entitled “System, Devices and Methods for Delivering a Flow of Oxygen,” filed May 25, 2022, the disclosure of which is incorporated by reference in its entirety.

### BACKGROUND

The embodiments described herein relate to systems, devices and methods for delivering a flow of an oxygen-containing gas. More specifically, embodiments described herein relate to a system and methods for providing real time adjustments to the flow and/or oxygen concentration of oxygen-containing gas to a patient.

Many patients admitted to the hospital are treated with supplemental oxygen. In the treatment of certain illnesses, such as COVID-19 related illnesses, oxygen is an important treatment tool, and the use of oxygen in both inpatient and outpatient settings has significantly increased over the last decade.

Delivery of oxygen is accomplished by flowing the oxygen-containing gas either through a nasal cannula that includes nasal prongs that are inserted into a patient's nostrils, or by the use of facial masks. One of the limiting factors for oxygen delivery is the patient's inspiratory flow rate, which can be characterized by the minute ventilation (the amount of air that enters the lungs per minute). If the minute ventilation is high due to high metabolic demands, the oxygen supplied through the nasal cannula can become insufficient. As a result, the entrainment of air from atmosphere increases and the nasopharyngeal reservoir is quickly depleted. This situation to some extent has been overcome by the advent of high flow delivery systems, which can provide gas flow of up to 60 L per minute by nasal cannula.

Although high flow nasal cannula oxygen delivery systems have provided improved oxygen delivery and can limit the likelihood of insufficient oxygen delivery, in many known methods, the flow rates are set arbitrarily without any real time knowledge of the patient's inspiratory flow rates or estimation of optimal flow rates to achieve the target oxygen saturation. Known methods of titration of oxygen can be a laborious process, which involves providers increasing the flow rates manually at set time intervals. With the increased need for oxygen related treatment in illnesses such as COVID-19, resources have been strained. Shortages of trained respiratory therapists and other medical providers has limited the ability to perform such tasks in a timely manner. As such, in many instances, the flow rate and/or oxygen concentration is set at an amount that is higher than is needed to avoid delivering insufficient oxygen.

Delivering excessive oxygen, however, can also have some disadvantages. For example, discomfort to the patient can occur due to large amounts of oxygen flow hitting the nasal mucus membranes, leading to drying and sometimes bleeding of nasal passages. In addition, excessive oxygen delivery can result in poor clearance of CO2, abdominal distention and risk of aspiration. Oxygen therapy is known to produce some amount of free radicals, and if the amount of oxygen delivered is excessive, the formation of free radicals (e.g., peroxynitrite) can cause damage to the lungs. Delivering excessive oxygen is also costly in that it causes waste of the oxygen.

In addition, the lack of knowledge of a patient's inspiratory flow rates during the delivery of oxygen may contribute to clinical deterioration of a patient. For example, without real-time knowledge of the patient's inspiratory flow rate, the delivery of oxygen to the patient can result in an insufficient flow of oxygen or provide too great of a flow of oxygen (which can produce excessive free radical formation and increased risks of lung injury). Inability to match gas flows to patient's inspiratory flow rates in a timely fashion can lead to worsening respiratory failure, fatigue, and ultimately respiratory arrest with poor outcomes, if invasive ventilation is not started on time.

In some outpatient settings, patients on supplemental oxygen can be started, for example, at a rate of 2-6 L/minute by nasal cannula. This flow rate may not match the actual day to day demands of the patient, which can lead to poor physical performance and deconditioning, and further vulnerability to aggravations of disease.

Delivering too much oxygen can also negatively impact the effectiveness of nebulized drugs. For example, delivery of nebulized medication with oxygen flow rates that do not match patient's inspiratory flow rates can result in a decrease in bioavailability of medication and loss of the drugs to the environment. For example, a recent study on porcine model demonstrated that if the ratio of gas flow to inspiratory flow is greater than one, less nebulized medication is delivered to the lungs than that which would be delivered if the if the ratio of gas flow to inspiratory flow is less than one.

Thus, a need exists for improvements in the delivery of oxygen and oxygen-containing gases to a patient that can provide real-time information and adjustment based on the inspiratory flow rate of the patient being treated.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, an apparatus for delivering a flow of oxygen-containing gas to an airway of a patient includes a nasal cannula having a nasal prong. The nasal prong includes a first end portion that defines an inlet opening, a second end portion that defines an outlet opening, and a middle portion between the first end portion and the second end portion. A side wall of the nasal prong defines a flow passage between the inlet opening and the outlet opening. The nasal prong is configured to be inserted within a nostril of a patient such that the outlet opening is disposed within the nostril. The inlet opening of the nasal prong is configured to be fluidically coupled to a support tube to deliver a flow of the oxygen-containing gas into the airway of the patient via the nasal prong. The side wall of the nasal prong defines a port at the second end portion of the nasal prong and in fluid communication with the flow passage. The port is configured to be fluidically coupled to a pressure sensor such that a series of pressure measurements within the flow passage of the nasal prong can be taken over a period of time during the delivery of the flow of the oxygen-containing gas.

In some embodiments, a system for delivering a flow of oxygen-containing gas to an airway of a patient includes a nasal cannula having at least one nasal prong. The nasal prong has a first end portion that defines an inlet opening and a second end portion that defines an outlet opening and has a side wall that defines a flow passage between the first end portion and the second end portion. The nasal prong is configured to be inserted within a nostril of a patient such that the outlet opening is disposed within the nostril. The side wall of the nasal prong defines a port. The nasal cannula is configured to be removably couplable to a source of the oxygen-containing gas such that the oxygen-containing gas can be delivered to the nasal cannula and into the airway of the patient via the nasal prong. The system includes a pressure sensor that is operably couplable to the nasal cannula and is configured to measure a pressure associated with the flow passage during a time period. The system includes a controller having a processor operatively coupled to the pressure sensor and that is configured to produce a pressure waveform characterizing the pressure as a function of time during the delivery of the oxygen-containing gas. The processor is configured to determine, based on the pressure waveform. an inspiratory flow rate of the patient. The processor is further configured to determine, based on the inspiratory flow rate, at least one of a tidal volume or a minute ventilation. The processor produces an output based at least in part on the determined inspiratory flow rate, the tidal volume, or the minute ventilation.

In some embodiments, the output includes a control signal that causes an adjustment to at least one of a percentage of oxygen within the oxygen-containing gas or a flow rate of the oxygen-containing gas being delivered into the nasal cannula.

In some embodiments, a method for delivering oxygen-containing gas to an airway of a patient includes providing a first flow of an oxygen-containing gas to a nasal cannula having a nasal prong inserted within a nostril of the patient. The nasal prong has a first end portion defining an inlet opening, a second end portion defining an outlet opening, and a flow passage between the inlet opening and the outlet opening. The first flow of the oxygen-containing gas has a first flow rate and a first percentage of oxygen within the oxygen-containing gas. A pressure is measured associated with the first flow of the oxygen-containing gas through the flow passage of the nasal prong via a pressure sensor over a time period. The pressure is communicated to a controller operatively coupled to the pressure sensor and a source of the oxygen-containing gas. The controller includes a processor. The method includes determining, at the processor, a pressure waveform characterizing the pressure as a function of time during the first flow of the oxygen-containing gas. Based on the pressure waveform, at the processor, at least one of an inspiratory flow rate of the patient, a tidal volume of the patient, or a minute ventilation of the patient is determined. A control signal is produced based on at least one of the inspiratory flow rate, the tidal volume, or the minute ventilation. The control signal is sent to the source of the oxygen-containing gas to adjust at least one of a percentage of oxygen within the oxygen-containing gas or a flow rate of the oxygen-containing gas being delivered into the nasal cannula via the first flow, thereby producing a second flow of the oxygen-containing gas.

In some embodiments, a device for delivering a flow of oxygen-containing gas to an airway of a patient includes a nasal cannula having at least one nasal prong. The nasal prong has a first end portion defining an inlet opening, a second end portion defining outlet opening, and a middle portion between the first end portion and the second end portion. A side wall of the nasal prong defines a flow passage between the inlet opening and the outlet opening. The nasal prong is configured to be inserted within a nostril of a patient such that the outlet opening is disposed within the nostril. The inlet opening of the nasal prong is configured to be fluidically coupled to a support tube to deliver a flow of the oxygen-containing gas into the airway of the patient via the nasal prong. The side wall of the nasal prong defines a set of ports at the second end portion of the nasal prong. The ports are configured to be fluidically coupled to a pressure sensor such that a pressure measurement associated with the flow passage of the nasal prong can be taken over a time period during the delivery of the flow of the oxygen-containing gas. The pressure measurement associated with a pressure at each port from the plurality of ports.

In some embodiments, a method for delivering oxygen-containing gas to an airway of a patient includes providing a flow of an oxygen-containing gas to a nasal cannula having at least one nasal prong inserted within a nostril of the patient. The nasal prong has a first end portion defining an inlet opening and a second end portion defining an outlet opening and defines a flow passage between the inlet opening and the outlet opening. The nasal prong defines a set of ports at the second end portion of the nasal prong. The flow of the oxygen-containing gas has a first flow rate and a first percentage of oxygen within the oxygen-containing gas. A pressure associated with the flow of the oxygen-containing gas through the flow passage of the nasal prong is measured via a pressure sensor. The measured pressure is based on a pressure associated with the flow passage at each port from the plurality of ports.

### BRIEF DESCRIPTION OF THE DRAWINGS

FIG. 1A is a schematic illustration of a flow regulated oxygen delivery system, according to an embodiment.

FIG. 1B is a schematic illustration of a nasal cannula of a flow regulated oxygen delivery system.

FIG. 2A is a schematic illustration of a nasal prong of the nasal cannula of FIG. 1B illustrating the flow of oxygen during inhalation by a patient.

FIG. 2B is a schematic illustration of a nasal prong of the nasal cannula of FIG. 1B illustrating the flow of oxygen during exhalation by a patient.

FIG. 3A is an illustrative plot showing pressure measured in the nasal prong of the cannula of FIGS. 1B during respiration.

FIG. 3B is an illustrative plot showing the respiratory flow rate determined based on the pressure waveform shown in FIG. 3A using methods according to an embodiment.

FIGS. 4A and 4B are each a schematic illustration of an alternative nasal prong of the nasal cannula of FIG. 1B, illustrating alternative locations for port(s) within the nasal prong.

FIGS. 4C and 4D are each a schematic illustration of an alternative nasal prong of the nasal cannula of FIG. 1B, illustrating alternative locations for ports within the nasal prong and varying wall thicknesses of the nasal prong.

FIG. 5 is a flowchart illustrating a method of delivering an oxygen-containing gas, according to an embodiment.

FIG. 6 is a schematic illustration of a nasal cannula of a flow regulated oxygen delivery system shown coupled to support tubes and being worn by a user, according to another embodiment.

FIG. 7 is an enlarged schematic illustration of the nasal cannula shown inserted within nostrils of the user.

FIG. 8A is a perspective view of the nasal cannula of FIG. 6.

FIG. 8B is a front view of the nasal cannula of FIG. 8A.

FIG. 9 is a top view of the nasal cannula of FIG. 8A, illustrating pressure sensor tubes coupled to the nasal cannula.

FIG. 10 is a rear view of the nasal cannula of FIG. 8A.

FIG. 11 is a cross-sectional view of the nasal cannula of FIG. 8A taken along line 11-11 in FIG. 9.

FIG. 12 is a cross-sectional view of a nasal prong of the nasal cannula of FIG. 8A.

FIG. 13 is a perspective view of a nasal prong of the nasal cannula of FIG. 8A.

FIG. 14A is a cross-sectional view of the nasal prong of FIG. 13.

FIG. 14B is a perspective cross-sectional view of the nasal prong of FIG. 13.

FIG. 14C is a cross-sectional view of the nasal prong of FIG. 13 illustrating example dimensions of the diameters and thicknesses of the sidewall of the nasal prong.

FIG. 15 is a cross-sectional view of the nasal prong of FIG. 13 illustrating a flow port channel of the nasal prong.

FIG. 16 is a perspective view of a nasal cannula, according to another embodiment.

FIG. 17 is a front view of the nasal cannula of FIG. 16.

FIG. 18 is a top view of the nasal cannula of FIG. 16.

FIG. 19 is a cross-sectional view of the nasal cannula of FIG. 16 taken along line 19-19 in FIG. 18.

FIG. 20 is an enlarged cross-sectional view of a nasal prong of the nasal cannula of FIG. 16.

FIG. 21 is a cross-sectional view of the nasal prong of FIG. 20 illustrating a flow port channel of the nasal prong.

FIG. 22 is a diagram of an example controller of a flow regulated oxygen delivery system.

FIGS. 23-25 are flow charts of various methods of controlling oxygen delivery to a patient according to embodiments.

FIGS. 26A and 26B show a perspective view (FIG. 26A) and a cross-sectional view (FIG. 26B) of an experimental mannequin used to simulate breathing through a patient's nostrils, to facilitate testing of the devices, systems, and methods described herein.

FIG. 27A is a plot of the cannula tip pressure as a function of time measured during a bench test with total cannula gas flow rate of 20 L/min and inspiration volume of 500 mL.

FIG. 27B is a plot of the calculated inspiratory flow rate as a function of time based on the pressure waveform.

FIG. 28A is a plot of the cannula tip pressure as a function of time measured during a bench test with total cannula gas flow rate of 20 L/min and inspiration volume of 1000 mL.

FIG. 28B is a plot of the calculated inspiratory flow rate as a function of time based on the pressure waveform.

FIG. 29 is a plot of the cannula tip pressure as a function of the patent flow rate as measured during a series of bench tests where the total cannula gas flow rate is 20 L/min.

FIG. 30A is a plot showing the measured cannula tip pressure as a function of the inspiratory flow rate from a series of bench tests with varying nasal prong gas flow rate.

FIG. 30B is a plot showing the measured cannula tip pressure as a function of the inspiratory flow rate with the flow rate being shifted to accommodate the different total cannula gas flow rates.

FIG. 31 is a perspective view of a nasal cannula, according to another embodiment.

FIG. 32 is a rear view of the nasal cannula of FIG. 31.

FIG. 33 is a top view of the nasal cannula of FIG. 31.

FIG. 34 is a cross-sectional view of the nasal cannula of FIG. 31, taken along line 34-34 in FIG. 33.

FIG. 35 is a side cross-sectional view of a portion of the nasal cannula of FIG. 31, taken along line 35-35 in FIG. 32.

FIG. 36 is a rear view of a nasal cannula, according to another embodiment.

FIG. 37 is a top view of the nasal cannula of FIG. 36.

FIG. 38 is a side view of the nasal cannula of FIG. 36.

FIG. 39 is a cross-sectional view of the nasal cannula of FIG. 36, taken along line 39-39 in FIG. 37.

FIG. 40 is a side cross-sectional view of a portion of the nasal cannula of FIG. 36, taken along the line 40-40 in FIG. 36.

FIG. 41 is a perspective view of a nasal cannula, according to another embodiment.

FIG. 42 is a rear view of the nasal cannula of FIG. 41.

FIG. 43 is a side view of the nasal cannula of FIG. 41.

FIG. 44 is a cross-sectional view of the nasal cannula of FIG. 41, taken along line 44-44 in FIG. 43.

FIG. 45 is a cross-sectional perspective view of a portion of the nasal cannula of FIG. 41, taken long line 45-45 in FIG. 42.

FIG. 46 is a side cross-sectional view of a portion of the nasal cannula of FIG. 41, taken along lines 46-46 in FIG. 42.

FIG. 47 is a side perspective view of a nasal prong, according to an embodiment.

FIG. 48 is a bottom perspective view of the nasal prong of FIG. 47.

FIG. 49 is a perspective view of a nasal cannula of a flow regulated oxygen delivery system shown coupled to support tubes, according to another embodiment.

### DETAILED DESCRIPTION

The systems, apparatus and methods described herein can be used to improve the delivery of oxygen-containing gas to a patient. The systems and apparatus described herein provide nasal delivery of oxygen-containing gas to a patient that is regulated based on any of the inspiratory flow rate, tidal volume, or minute ventilation as determined based on one or pressure measurements. More specifically, the systems and apparatus described herein can be used to deliver a flow of an oxygen-containing gas to a patient via a nasal cannula that can be coupled to a pressure sensor and used to measure pressures within a flow passage of the nasal cannula. The pressure measurements can be used to determine inspiratory flow rates, tidal volume, and/or minute ventilation of the patient in real time during the delivery of the oxygen-containing gas, which can be used to automatically make adjustments to the flow rate and/or to the percentage of oxygen within the oxygen-containing gas during the treatment. The pressure measurement data can be delivered to a computer processor (e.g., microcontroller), and the computer processor can analyze pressure waveform (as a function of time) within flow passage of the nasal cannula and determine an inspiratory flow rate of the patient. The processor can then send the information to a gas flow control valve which, in turn, can automatically adjust the flow rate of the oxygen-containing gas and/or adjust a percentage of oxygen within the oxygen-containing gas being delivered to the patient based on the information (e.g., inspiratory flow rate of the patient). In this manner. the systems and methods described herein can match gas flow (and amount of oxygen delivered) to the patient's inspiratory flow rates. Flow rates can be regulated between, for example, two liters per minute to as high as 60-80 liters per minute. The systems and methods described herein can also enhance delivery of nebulized medication and improve bioavailability. By analyzing the basal flow rates and waveform pattern of patients flow dynamics (described in more detail below) the computer processor can cause the delivery of the oxygen-containing gas in a decelerating fashion during the inspiratory time interval. The system can provide end-tidal CO₂ (ETCO2), oxygen saturation and minute ventilation information to medical care providers and assist in early transition to a higher modality of ventilation if desired.

The systems and apparatus described herein provide multiple use modes, including an autonomous mode, a manual mode and a code mode. The autonomous mode is fully controlled by a computer processor. In this mode the system can titrate oxygen and provide the desired oxygen flow and concentration based on the patient's saturation inputs and metabolic demands by regulating the flow and concentration based on the patient's inspiratory flow rate, tidal volume, and/or minute ventilation. The embodiments described herein can automatically sense a patient's clinical decline based, for example, on changes in the inspiratory flow rate, and can transition from a low flow to a high flow of oxygen delivery. In the manual mode (used when oxygen saturation signal from the patient is not valid), flows can be matched the patient's demand; however, oxygen titration would be completely under the control of the care provider. With the code mode, the system provides 100% oxygen at maximum flows, and subsequently can go into autonomous mode and use the least needed flows and oxygen concentration to maintain the desired saturations.

The systems and apparatus described herein can provide flow rates of oxygen to a patient that can match the patient's demand, reduce discomfort, abdominal distention, oxygen toxicity, improve CO2 clearance, and provide real-time information to care givers to better control and provide a desired percentage of oxygen within the oxygen-containing gas delivered to the patient. In addition, enhanced nebulized medication delivery can be accomplished using the flow-regulated oxygen delivery system described herein by delivering the nebulized medication on the dry side of the humidifier/gas mixer and providing medications at a patient's inspiratory flow rates, thus improving bioavailability and treatment outcomes.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

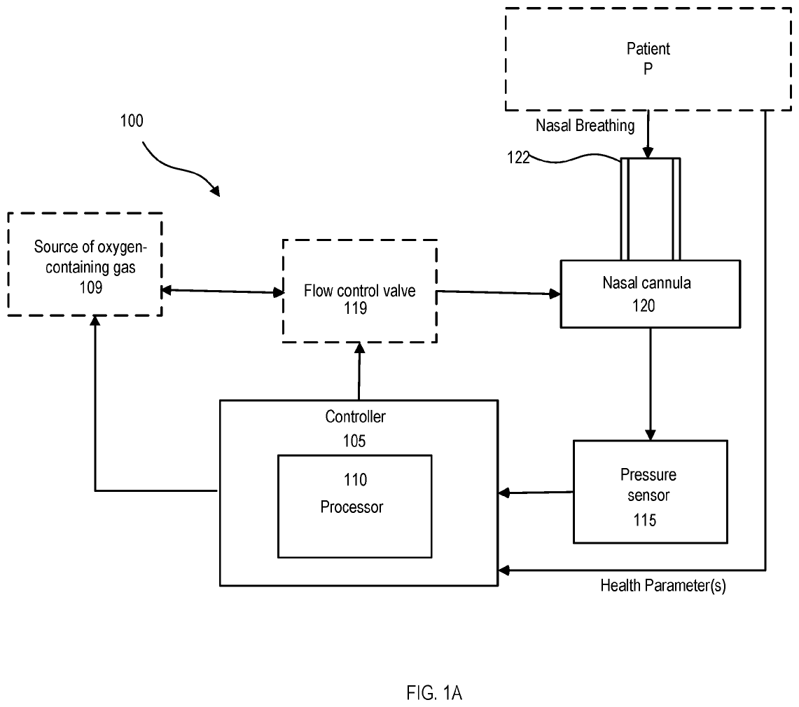

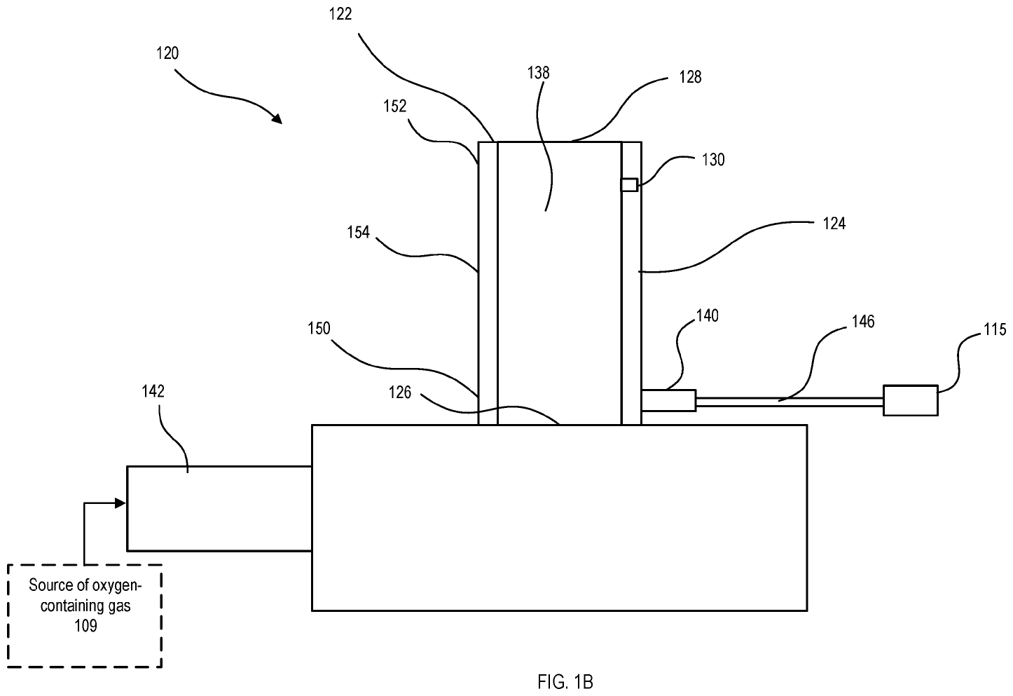

FIGS. 1A and 1B are schematic illustrations of a flow regulated oxygen delivery system 100 (also referred to as “flow regulated system” or “delivery system” or “system”), according to an embodiment. The system 100 can be used to deliver an oxygen-containing gas to a patient P and provide real-time information related to the patient's inspiratory flow rate. tidal volume, and/or minute ventilation during the delivery of the gas. With this information, the system can make adjustments to the flow rate of the oxygen-containing gas and/or to the amount or fraction of oxygen within the oxygen-containing gas in real-time during the delivery of the gas.

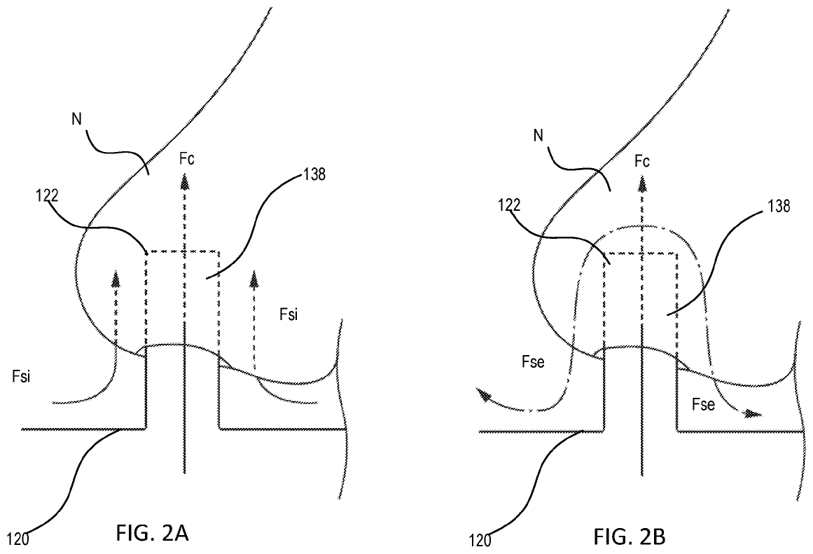

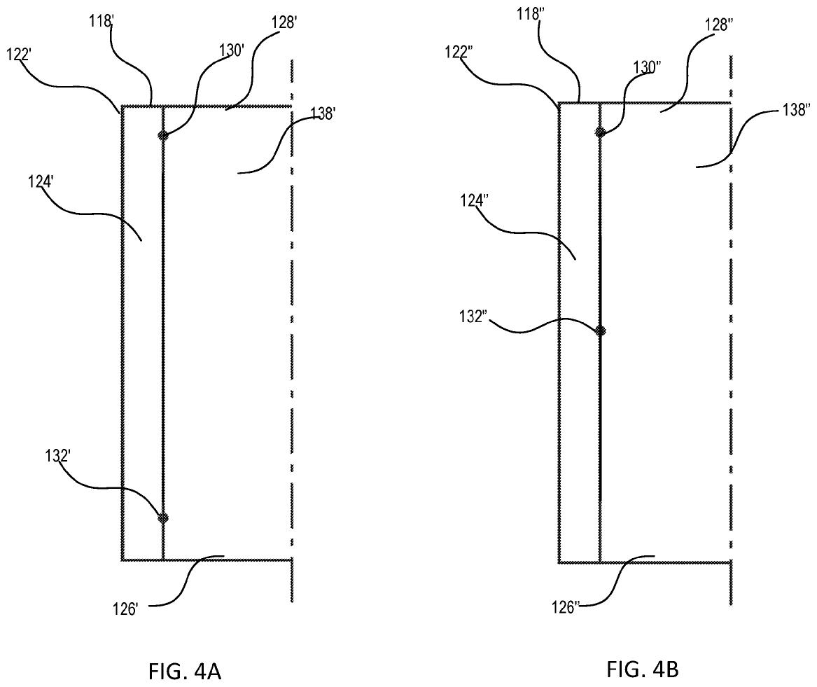

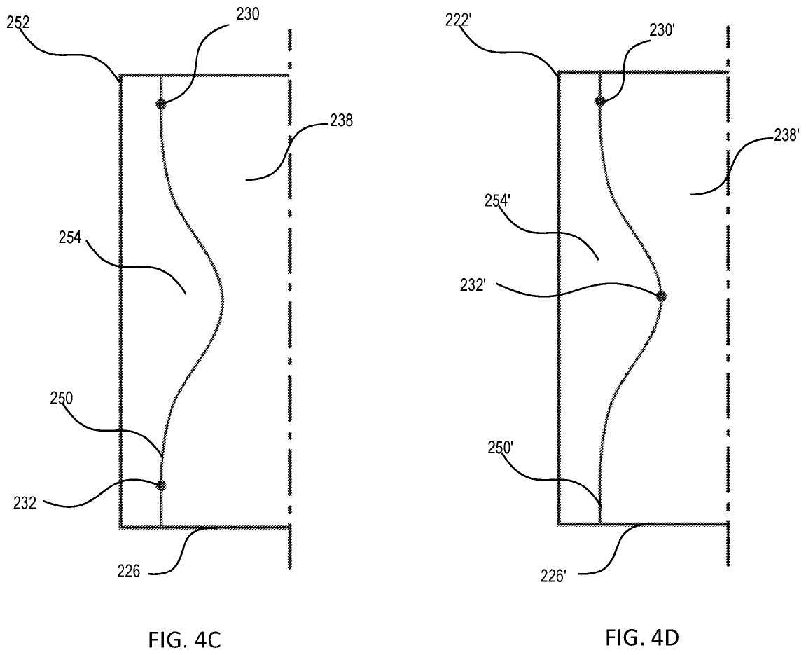

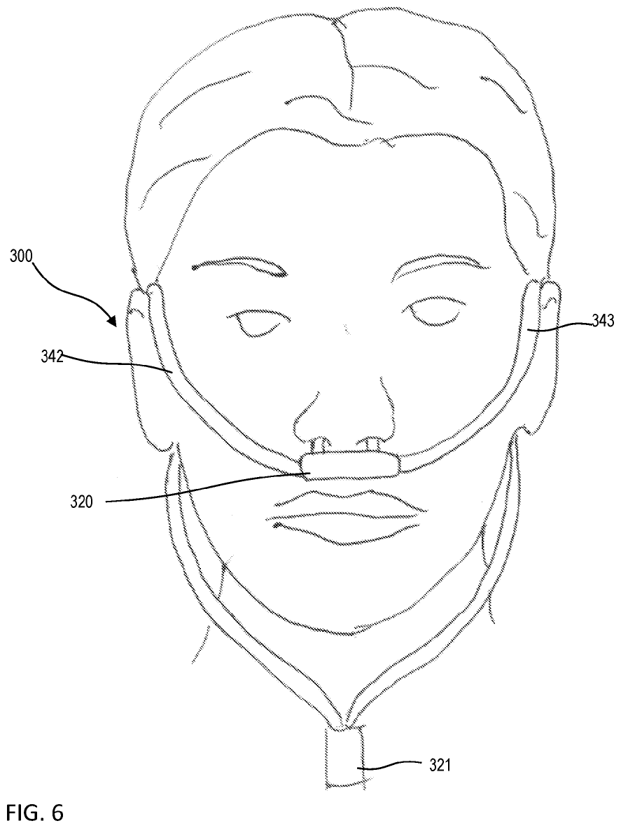

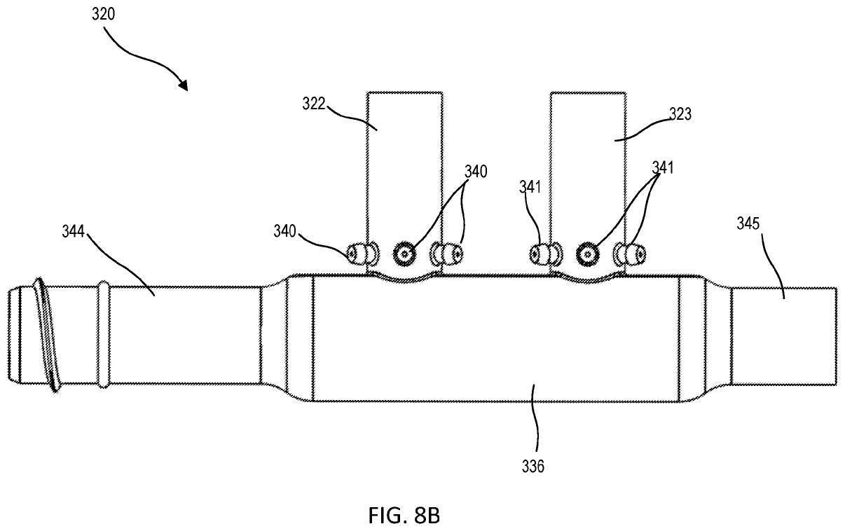

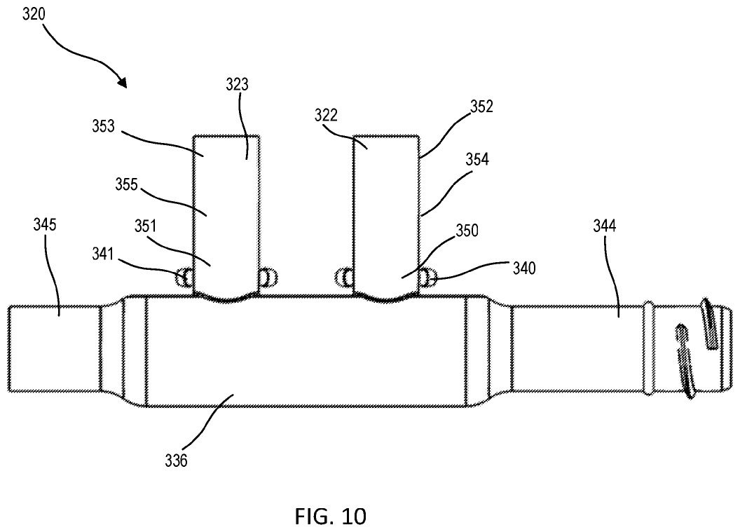

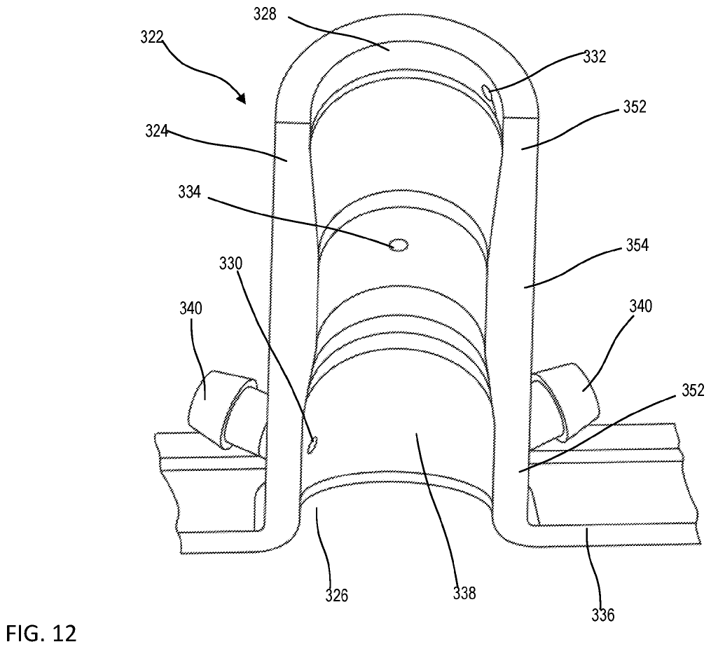

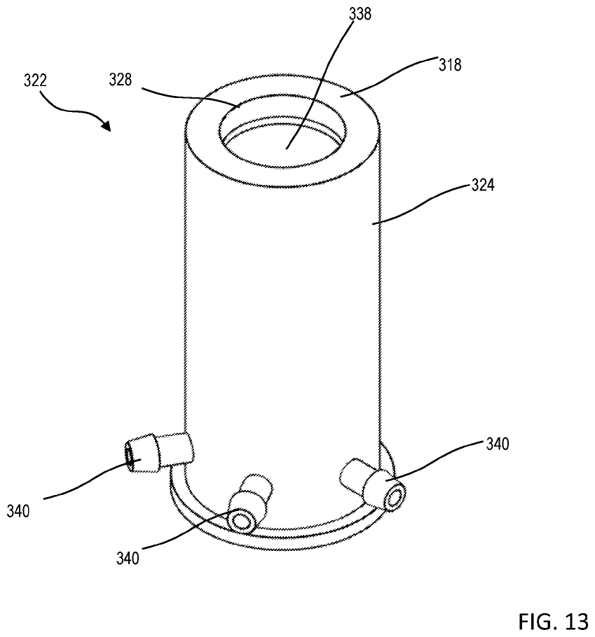

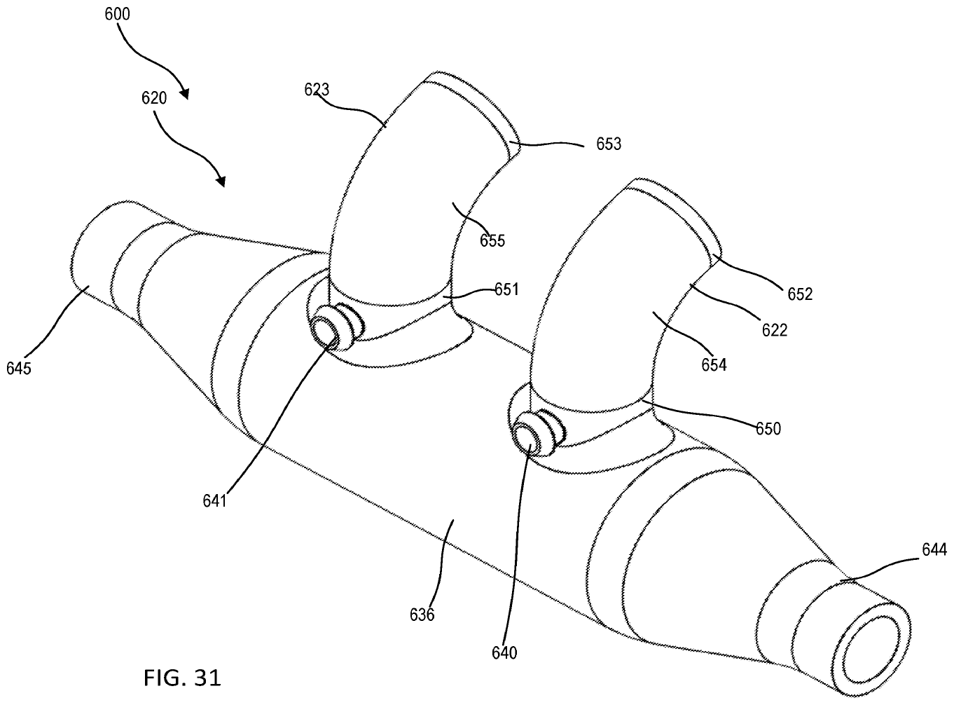

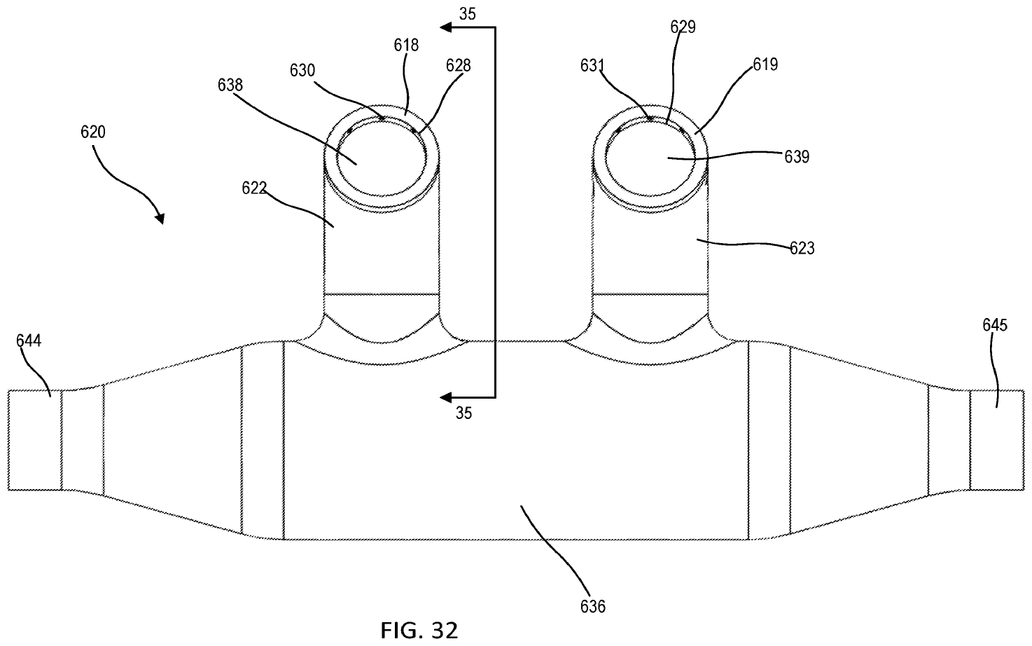

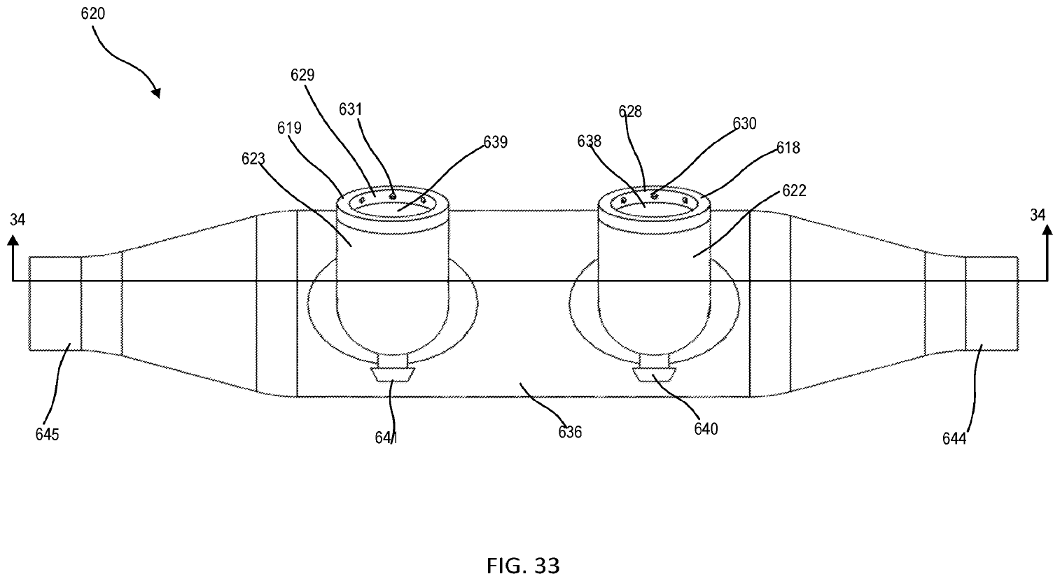

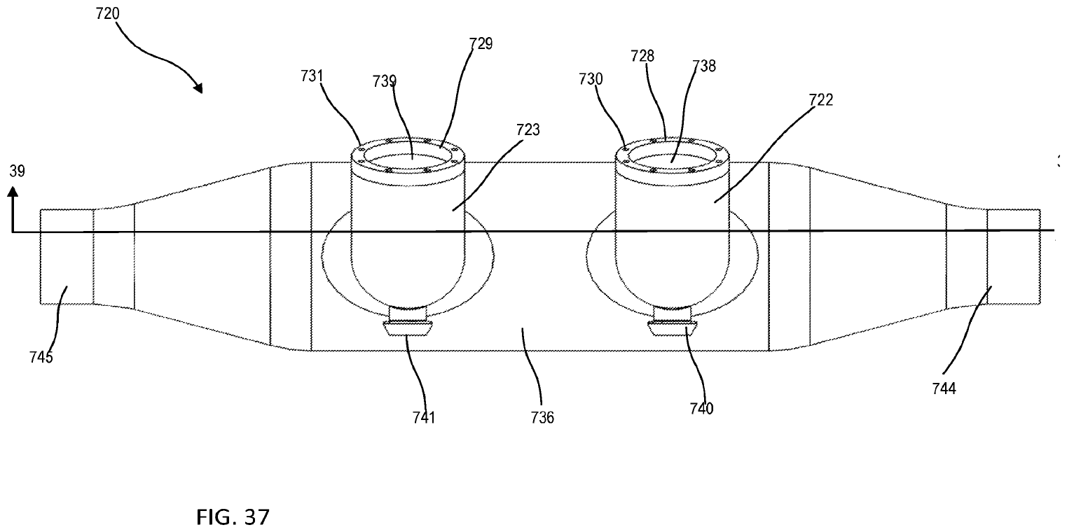

The system 100 includes a nasal cannula 120, a pressure sensor 115 and a controller 105. The nasal cannula 120 includes at least one nasal prong 122 that has a first end portion 150 that defines an inlet opening 126, and a second end portion 152 that defines an outlet opening 128, and a middle portion 154, as shown in FIG. 1B. The nasal prong 122 includes a side wall 124 that defines a flow passage 138 between the first end portion 150 and the second end portion 152. For example, the flow passage 138 is defined by an inner surface of the side wall 124. The nasal prong 122 is configured to be inserted within a nostril of a patient such that the outlet opening 128 is disposed within the nostril N of a user, as shown for example, in FIGS. 2A and 2B. The side wall 124 of the nasal prong 122 defines a port 130 within the flow passage 138 at the second end portion 128 of the nasal prong 122. In other embodiments, the port 130 can be at any location within the flow passage 138 or at an end surface near the outlet of the flow passage 138 (similar to the arrangement shown, for example, in the nasal cannula 720 described herein). The nasal cannula 120 can optionally include a second nasal prong (not shown) that can be inserted within a second nostril of the patient. Such an embodiment is described below with reference to FIGS. 6-15. Further, the nasal prong 122 can include a different number of ports, for example, two, three or more ports as described below with reference to FIGS. 4A-4D and with reference to FIGS. 6-15. Moreover, the nasal prong 122 (and any of the nasal prongs described herein) can include any number of ports at a similar axial location, such as the configurations described with reference to FIGS. 31-41.

The nasal cannula 120 can be removably coupled to a source of oxygen-containing gas 109 (see e.g., FIG. 1A) such that the oxygen-containing gas can be delivered to the nasal cannula 120 and into the airway of a user/patient via the nasal prong 122. The pressure sensor 115 can also be operably coupled to the nasal cannula 120 and can be used to measure pressures within or associated with the flow passage 138 of the nasal prong 122. For example, the pressure sensor 115 can be operatively coupled to the port 130 of the nasal prong 122 via a sensor coupler 140 and a pressure sensor tube 146. The port 130 allows for pressure measurements to be taken within the flow passage 138 via the pressure sensor 115 during delivery of the oxygen-containing gas to the nasal cannula 120. For example, the port 130 can be in fluid communication with the pressure sensor 115 via a fluid port channel (not shown) defined in the sidewall 124 of the nasal prong 122. The pressure sensor 115 can be a single sensor that communicates with only port 130 or can be one sensor that is multiplexed to measure pressure at multiple ports as described herein for alternative embodiments. With multiplexing, the single sensor can be “switched” to measure pressure at each of the different ports.

Referring to FIG. 1B, the port 130 and fluid port channel (not shown) can be in fluid communication with the sensor coupler 140 that is coupled to the pressure sensor 115 via a pressure sensor tube 146. As also shown in FIG. 1B, the flow passage 138 is in fluid communication with a support tube 142. The support tube 142 can be fluidically coupled to the source of oxygen-containing gas such that the gas can be delivered to the nasal cannula 120 via the support tube 142. The support tube 142 can be a flexible tubing that can be used to support the nasal cannula 120 on the head of the patient. For example, the support tube 142 can extend around an ear of the patient as shown in FIG. 6 and described in more detail below.

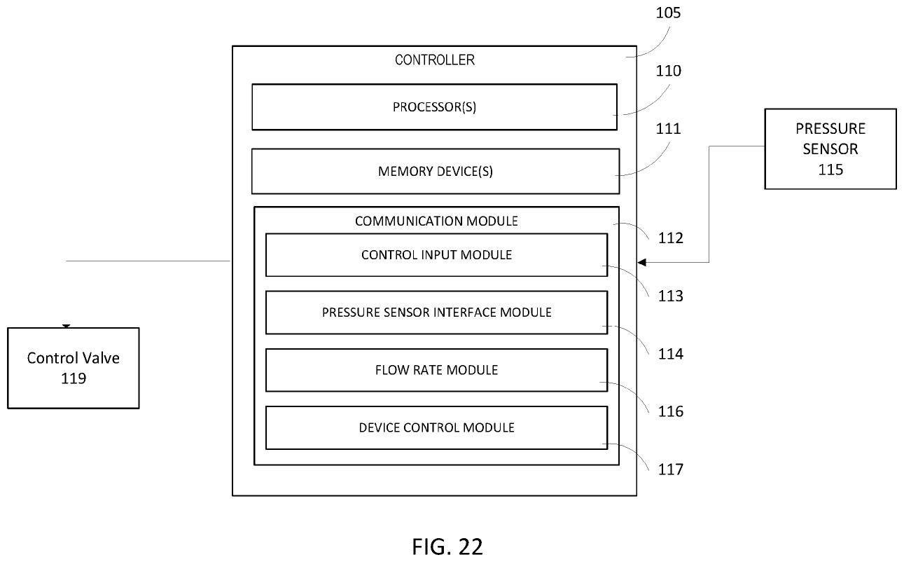

The controller 105 can be any suitable controller containing the hardware (e.g., the processor 110, a memory component), firmware, and/or software to perform any of the methods and functions described herein. For example, in some embodiments, the controller 105 can include one or more optional modules or a memory as described below with reference to FIG. 22. As shown in FIG. 1A, the controller has a processor 110 that is operatively coupled to the pressure sensor 115 and is configured to receive a series of pressure measurements from the pressure sensor 115, manipulate or process the pressure measurements (e.g., to produce a pressure waveform), determine an inspiratory flow rate based on the pressure measurements, and produce an output to control delivery of the oxygen-containing gas to the patient. Specifically, the processor 110 can produce an output based at least in part on any of the determined inspiratory flow rate, a tidal volume calculated based on the inspiratory flow rate, or a minute ventilation based on the inspiratory flow rate over time. For example, the output produced by the processor 110 can include a control signal that causes an adjustment to at least one of a percentage of oxygen within the oxygen-containing gas or a flow rate of the oxygen-containing gas being delivered into the nasal cannula 120. In some embodiments, the control signal is sent to the source of the oxygen-containing gas 109, which can make adjustments to the flow of the oxygen-containing gas. In some embodiments, the control signal can be sent to a flow control valve 119 that is coupled to the source of the oxygen-containing gas 109 and coupled to the nasal cannula 120 to deliver the oxygen-containing gas to the nasal cavity of the patient. In some embodiments, the flow control valve 119 is included with or coupled to a humidifier (not shown) through which the gas flows before being delivered to the nasal cannula 120. In some embodiments, the source of oxygen-containing gas 109, the humidifier and the flow control valve 119 are all included in the same device.

With information related to the patient's inspiratory flow rate determined by the processor 110, adjustments can automatically be made to the flow of the oxygen-containing gas in real-time during the delivery of the oxygen-containing gas. Thus, the amount of oxygen delivered can be associated with (or matched) to the patient's inspiratory flow, and therefore the patient's metabolic demands. In this manner, the system 100 and methods described herein can ensure that a sufficient amount of oxygen is being delivered while also limiting the likelihood of delivering excess oxygen (which is both wasteful and potentially detrimental to the patient, as described herein). By determining the inspiratory flow rate based on the pressure measurements from the nasal cannula 120 used to deliver the oxygen-containing gas, the system 100 does not require additional equipment (e.g., a cumbersome flow meter) to titrate oxygen and provide the desired oxygen flow. Moreover, although the system 100 can receive one or more additional health parameters related to the patient (e.g., measured oxygen saturation SaO2 or SpO2 measured via pulse oximetry) as input to control the oxygen delivery, adjusting the flow and/or oxygen concentration of the oxygen-containing gas based on the patient's inspiratory flow rate as determined from the cannula pressure measurements provides an improved level of control. Specifically, the methods described herein can include controlling the amount of oxygen delivered based on the patient's inspiratory flow rate as a first (or an inner) control loop to effectively match the flow rate of oxygen-containing gas with the patient's inspiratory flow rate, while also including a second (or an outer) control loop based on the measured oxygen saturation. Such methods can improve the likelihood of delivering the desired amount of oxygen in real time by relying on the first (or inner) control loop (based on the patient's inspiratory flow rate), which has a very low lag time, and also the second (or outer) control loop (based on the SaO2 or SpO2), which is the desired end parameter.

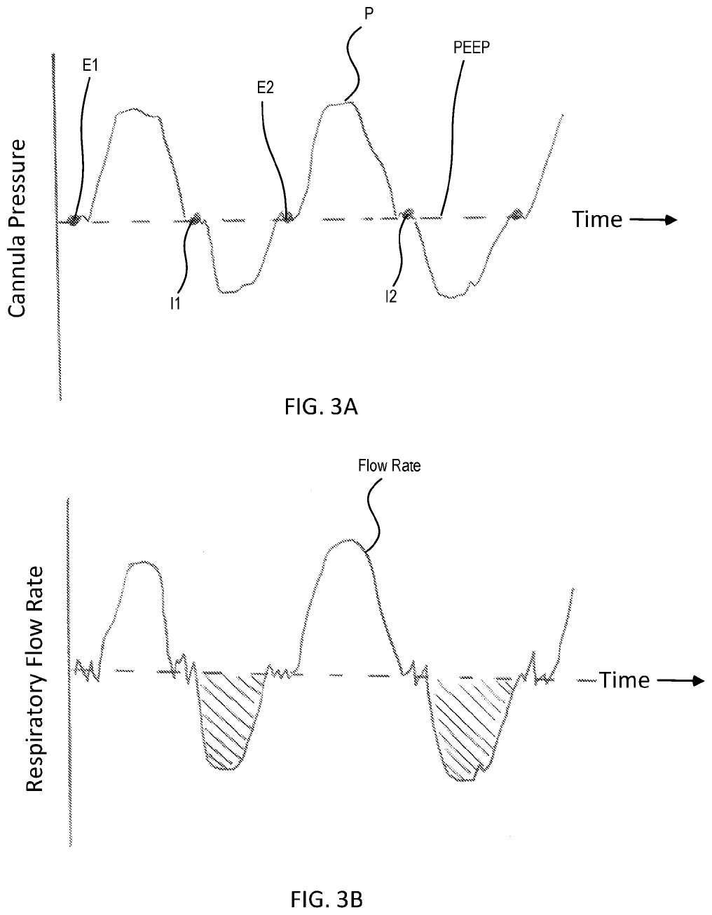

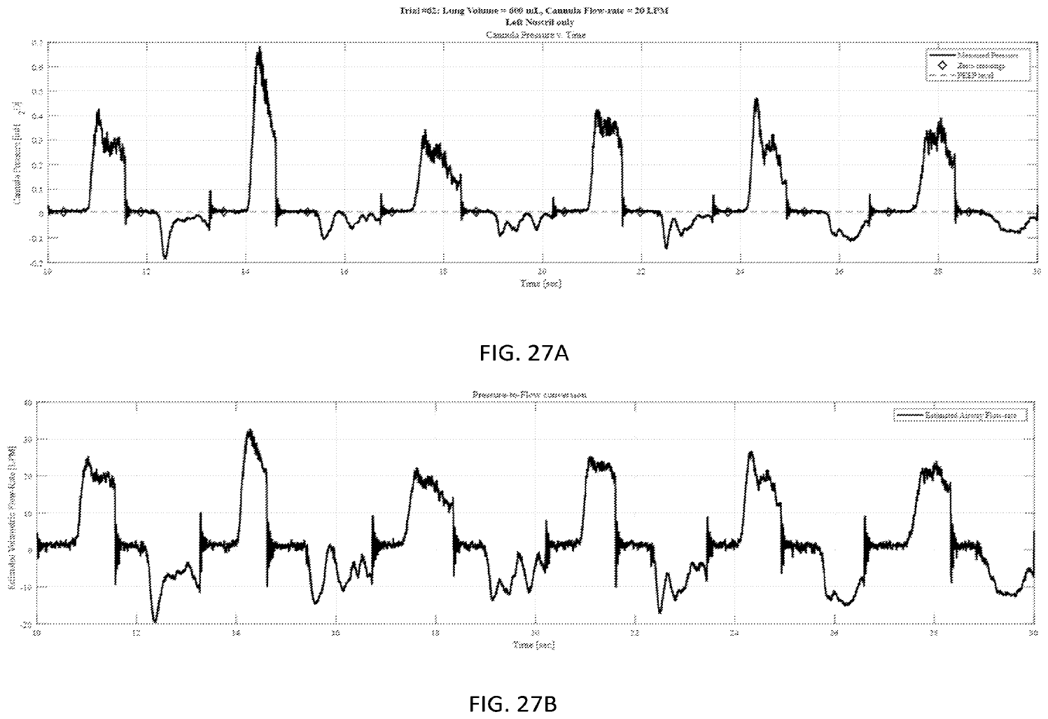

The methods of delivering an oxygen-containing gas are illustrated by FIGS. 2A and 2B, which are schematic illustrations showing gas flowing through the nasal prong 122 and into the nasal cavity of patient (FIG. 2A shows an inhalation and FIG. 2B shows an exhalation) and FIGS. 3A-3C, which show illustrative, schematic plots of data that can be measured from the nasal cannula 120 (actual data from bench testing is provided in FIGS. 27A-30B). As shown in FIGS. 2A and 2B, in use the oxygen-containing gas flows through the nasal prong 122 and into the nasal cavity of the patient with a flow rate Fc. The flow rate Fc is associated with the delivery flow rate of the system 100 and into the nasal cannula 120 and nasal prong 122, and in some embodiments is maintained at a steady value through at least one respiratory cycle. There may also be a flow of air being inhaled by the patient in the space surrounding the nasal prong 122 at a flow rate Fsi, as shown in FIG. 2A, and a flow of air being exhaled by the patient in the space surrounding the nasal prong 122 at a flow rate of Fse, as shown in FIG. 2B. The flow rate of Fsi and Fse are dependent on many different factors, including (but not limited to) the flow rate Fc of the oxygen-containing gas, the anatomical features of the patient (e.g., the size of nostril, which can impact the size of the space surrounding the nasal cannula 120), the positioning of the nasal prong 122 within the patient's nostril, and the breathing pattern of the patient. Additionally, although the flow rate Fsi is shown as being in the inhalation direction (into the nasal cavity), in some situations (e.g., if the flow rate Fc is sufficiently high), the flow rate Fsi will be in the exhalation direction (out of the nasal cavity). Because the Fsi and Fse are different for each patient and situation, and are not easily measured, the actual flow rate of gas being inhaled and exhaled by the patient is not suitable for direct measurement in real time. The current system 100, nasal cannula 120, and methods described herein, however, can determine the inspiratory flow rate (the amount of gas being inhaled into the patient's lungs) based on the pressure within the flow passage 138 of the nasal prong 122 as measured at the port 130.

FIG. 3A shows a plot of an illustrative pressure waveform based on measurements taken within the flow passage 138 of the nasal prong 122 at port 130 as a function of time. The representative plot shows the pressure during a time period that includes two respiratory cycles (each including an exhalation event and an inhalation event). As shown, the cannula pressure P increases during an exhalation event due to the back pressure exerted by exhalation gas flow at the cannula exit opening 128 and into the flow passage 138. The cannula pressure P decreases during an inhalation event due to the reduced pressure (suction) at the cannula exit opening 128 caused by the inhalation flow. The dwell period between the exhalation and inhalation events are shown as regions of relatively limited change in the pressure P. The beginning of the exhalation events (which can also be the end of the inhalation events) are identified as E1 and E2 and the beginning of the inhalation events (which can also be the end of the exhalation events) are identified as I1 and I2. The baseline pressure at the beginning and end of the exhalation and inhalation events is referred to as the positive end-expiratory pressure (PEEP). The PEEP is influenced by the flow rate Fc of the oxygen-containing gas and increases with increased flow rate Fc and decreases with decreased flow rate Fc. Thus, depending on the flow rate Fc, the magnitude of the pressure waveform during an inhalation event can vary. For example, when the flow rate Fc is relatively low (e.g., less than 5 L/min). the measured pressure during inhalation events will generally be below atmospheric pressure. In contrast, when the when the flow rate Fc is relatively high (e.g., between 30 and 40 L/min), the measured pressure during inhalation events will be higher and may even remain slightly positive throughout the inhalation event.

The inventors have discovered that notwithstanding the influence of the flow rate Fc, the cannula pressure P can be correlated to the inspiratory and expiratory flow rate and thus FIG. 3B shows an illustrative plot of the respiratory flow rate as a function of time. As described in more detail herein, the respiratory flow rate can be determined by the processor 110 based on any suitable technique. For example, in some embodiments, the processor 110 can determine the respiratory flow rate using a curve fit based on empirical data that includes the measured pressure for various gas flow rates Fc and the resulting respiratory flow rates. In other embodiments, the processor 110 can determine the respiratory flow rate using a series of “lookup” tables. In yet other embodiments, the system can be calibrated for a specific application (e.g., the cannula fitted to a specific patient), and the processor 110 can determine the patient's respiratory flow rate based on the calibration data.

In some embodiments, the processor can further manipulate the respiratory flow rate waveform shown in FIG. 3B to determine a tidal volume of the patient or a minute ventilation of the patient. For example, in some embodiments, the processor 110 can use any suitable analysis technique to identify the beginning and ending of each inhalation and each exhalation event. Then, each event can be numerically integrated between the beginning and end points to determine the total volume exchanged during the specific event. Thus, in some embodiments, the processor 110 can numerically integrate the inspiratory flow rate curve for each inhalation event to determine the tidal volume for each inhalation event (see the cross-hatched shading in the inhalation events in FIG. 3B as a representative of the area under the curve that is produced by the numerical integration described herein). The total inspiratory volume can be summed over a desired time period (e.g., one minute) to determine a minute ventilation value.

With the information related to the inspiratory flow rate, the tidal volume, and/or the minute ventilation of the patient, the system 100 can make automatic adjustments to the flow rate and/or to the amount of oxygen within the oxygen-containing gas. For example, the processor 110 can produce an output based at least in part on any or all of the determined inspiratory flow rate, the tidal volume, and/or the minute ventilation. The output produced by the processor 110 can include a control signal that causes an adjustment to at least one of a percentage of oxygen within the oxygen-containing gas or a flow rate of the oxygen-containing gas being delivered into the nasal cannula based at least in part on the determined inspiratory flow rate. In some embodiments, the control signal can be sent to a flow control valve 119 that is coupled to the source of the oxygen-containing gas and coupled to the nasal cannula 120 to deliver the oxygen-containing gas to the nasal cavity of the patient. The control valve 119 can then automatically adjust the flow rate and/or percentage of oxygen being delivered to the patient in real-time. In this manner, the system 100 can match the flow rate of oxygen-containing gas and/or the concentration of the oxygen therein with the patient's inspiratory flow rate.

As described above, in some embodiments, the processor 110 can receive input including data related to a health parameter associated with the patient. In such a case, the processor 110 can produce the control signal based at least in part on any of the determined inspiratory flow rate, the tidal volume, the minute ventilation and the health parameter(s). For example, as described in more detail herein, the additional health parameter can be the measured oxygen saturation (e.g., as measured via pulse oximetry). In some embodiments, the processor 110 can produce a first control signal to adjust the oxygen concentration within the oxygen-containing gas based on the measured oxygen saturation (e.g., increasing the oxygen concentration if the oxygen saturation is below a target level). The processor 110 can produce a second control signal to adjust the flow rate of the oxygen-containing gas (based on the cannula pressure measurements) to match the patient's inspiratory flow rate. In some embodiments, one or more control signals is received by the flow control valve 119 such that the adjustment the percentage of oxygen within the oxygen-containing gas or the flow rate of the oxygen-containing gas being delivered into the nasal cannula 120 is based at least in part on the determined inspiratory flow rate and the health parameter(s).

Although described as being performed in the processor 110, in some embodiments, the controller 105 can include any suitable components to perform the functions described herein. For example, in some embodiments, the controller 105 can include a pressure sensor interface module 114 (see, e.g., FIG. 22) that can receive the signal from the sensor 115 and perform desired signal filtering and analysis of the shape of the pressure waveform to accurately identify the beginning and ending of each exhalation and inhalation event. In some embodiments, the controller 105 can include a flow rate module 116 (see. e.g., FIG. 22) that can manipulate the pressure waveform to determine the inspiratory (or expiratory) flow rate of a patient. For example, in some embodiments, the controller 105 can include a memory device 111 that stores one or more calibration tables or parameters (e.g., calibration coefficients). The flow rate module 116 can access the calibration tables or parameters, can receive an input related to the flow rate Fc of the oxygen-containing gas, and manipulate the pressure waveform to determine the patient's inspiratory flow rate. In some embodiments, the controller 105 can include a device control module 117 (see, e.g., FIG. 22) that can produce one or more control signals to adjust a flow rate of the oxygen-containing gas or adjust a concentration of oxygen within the gas. The device control module 117 can include any suitable control parameters and can execute any desirable control algorithms to produce the desired level of control of oxygen content within and/or flow rate of the oxygen-containing gas.

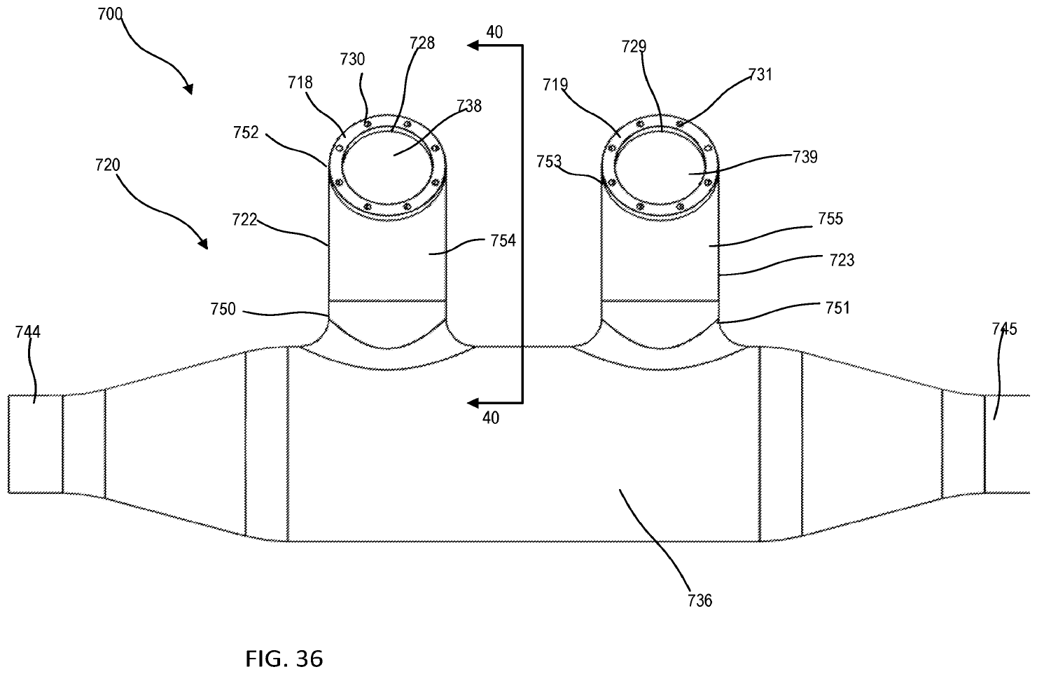

As described above, the nasal prong 122 includes the first end portion 150 and the second end portion 152 and a middle portion 154 between the first end portion 150 and the second end portion 152. The nasal prong 122 also includes the port 130 at the first location within the nasal prong 122. In alternative embodiments, the nasal prong 122 can include any suitable number of ports at any suitable location within or adjacent to the flow passage 138. For example, in some embodiments, the nasal prong 122′ can include a second port 132′ defined by the sidewall 124′ at or near the first end portion 150′ as shown schematically in FIG. 4A. In other embodiments, a nasal prong 122″ can include a second port 132″ at or near the middle portion 154″, as shown schematically in FIG. 4B. In yet other embodiments, the nasal prong can include one or more ports located at an end surface near the outlet of the flow passage (similar to the configuration shown in FIGS. 36-40). The positioning of the first port 130 at or near the second end portion 152 enables the pressure within the flow passage 138 to be measured at or near the outlet opening 128 of the nasal prong 122. In some embodiments, the port 130 is defined by the side wall 124 at a distance from an end surface 118 of the nasal prong 122 of not more than four times a diameter of the port 130. In some embodiments, the port 130 is defined by the side wall 124 at a distance from an end surface 118 of the nasal prong 122 of not more than two times a diameter of the port 130. In some embodiments, the port 130 is defined by the side wall 124 at a distance from an end surface 118 of the nasal prong 122 of between about one and three times a diameter of the port 130. The close proximity of the first port 130 to the end surface 118 of the nasal prongs 122 provides for a pressure measurement closer to the outlet opening 128 of the nasal prong 122, which can result in a more accurate determination of the inspiratory flow rate of the patient based on the pressure measurements. For example, the location of the first port 130 can be selected to maximize the impact of the back pressure produced by the patient's respiration (i.e., reduce the damping of the nasal pressure that can be caused by the gas flow Fc).

In some alternative embodiments, the side wall 124 can also have varying thickness such that an inner diameter of the flow passage 138 varies between the first end portion 150 and the second end portion 152 as shown, for example, in the schematic illustrations of FIGS. 2C and 2D. For example, FIGS. 4C and 4D illustrate a nasal prong 222, 222′ that can have a first inner diameter at a first end portion 250, 250′ and a second inner diameter at a second end portion 252, 252′ that are each greater than an inner diameter at a middle portion 254, 254′. See also, for example, cannula 320 described below with reference to FIGS. 6-15, which illustrates the varying diameter and wall thickness of the nasal prongs 322 and 323 of the nasal cannula 320. This construction with a varying inner diameter provides for a venturi effect within the flow passage 238, 238′. FIGS. 4C and 4D also illustrate a nasal prong having two ports. FIG. 4C illustrates the nasal prong 222 having a first port 230 defined by a sidewall 224 at the second end portion 252 and a second port 232 defined by the sidewall 224 at the first end portion 250. As shown in FIG. 4D, the nasal prong 222′ has a first port 230′ defined by a sidewall 224′ at the second end portion 252′ and a second port 232′ defined at the middle portion 240′. The additional ports can provide for a redundancy in the pressure measurement, thereby improving the accuracy of the inspiratory flow rate determined based on the pressure measurement. The venturi design can also provide an approximate measurement of the flow rate Fc of the oxygen-containing gas within the passageway.

Although not shown in FIGS. 1A and 1B, in some embodiments, the nasal cannula 120 can include a second nasal prong (not shown) that can be inserted in a second nostril of the patient and used to deliver a flow of oxygen-containing gas to the nasal cavity of the patient. The second nasal prong can include the same or similar features as the nasal prong 122. For example, the second nasal prong can include one or more ports defined in a sidewall that can be operatively and fluidically coupled to a pressure sensor such that pressures within a flow passage of the second nasal prong can be measured and used to determine an inspiratory flow rate of the patient. Such an embodiment is shown and described below which reference to FIGS. 6-15.

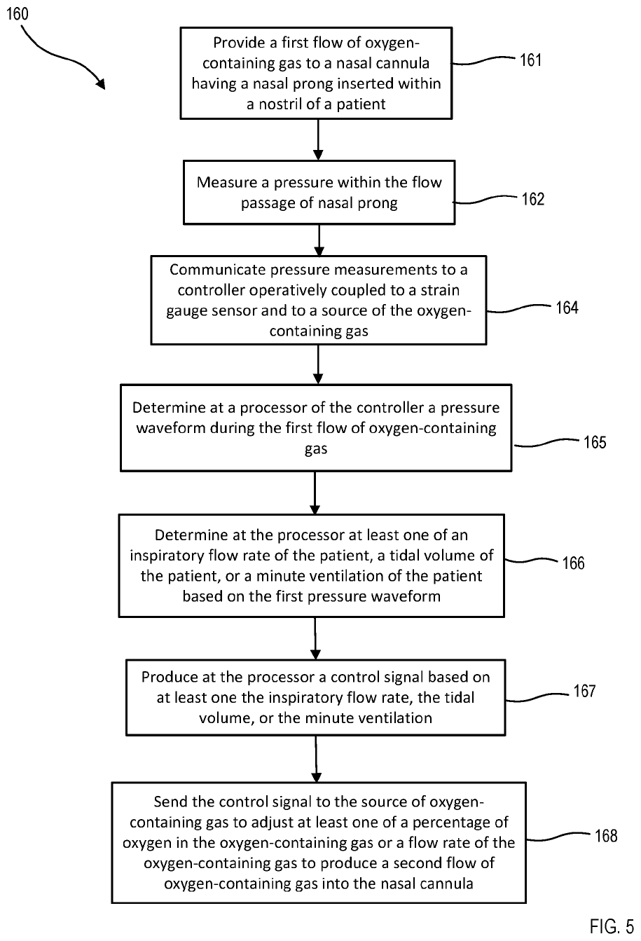

FIG. 5 is a flow diagram of an example method of using a flow regulated nasal delivery system for delivering oxygen-containing gas to an airway of a patient as described herein. The method 160 includes at 161 providing at a first flow rate, an oxygen-containing gas to a nasal cannula (e.g., 120) having a nasal prong (e.g., 122) inserted within a nostril of the patient. As described herein. the nasal prong (e.g., 122) has a first end portion defining an inlet opening, a second end portion defining an outlet opening, and a flow passage between the inlet opening and the outlet opening. The first flow of an oxygen-containing gas has a first flow rate and a first percentage of oxygen within the oxygen-containing gas. At 162, a pressure is measured over a time period within (or near) the flow passage of the nasal prong via a pressure sensor (e.g., 115). At 164, the pressure measurements are communicated to a controller (e.g., 105) operatively coupled to the pressure sensor and a source of the oxygen-containing gas. The controller includes a processor (e.g., 110) and at 165, the processor determines a pressure waveform characterizing the pressure measurements as a function of time during the first flow of the oxygen-containing gas. At 166, based on at least the pressure waveform, at the processor, at least one of an inspiratory flow rate of the patient a tidal volume of the patient, or a minute ventilation of the patient is determined. At 167, a control signal is produced at the processor based at least on at least one of the inspiratory flow rate, the tidal volume, or the minute ventilation. At 168, the control signal can be sent to the source of the oxygen-containing gas to adjust at least one of a percentage of oxygen within the oxygen-containing gas or a flow rate of the oxygen-containing gas being delivered into the nasal cannula via the first flow, thereby producing a second flow of oxygen-containing gas.

FIGS. 6-15 illustrate portions of a flow regulated nasal delivery system (also referred to as “flow regulated system” or “delivery system” or “system”), according to another embodiment. The system 300 can be used to deliver an oxygen-containing gas to a patient and provide real-time information related to the patient's inspiratory flow rate during the delivery of the gas and can include the same or similar features and function the same or similar as the system 100 described above. Thus, some features are not shown and described with reference to this embodiment. As with the previous embodiment, the system 300 can make automatic adjustments to the flow rate of the oxygen-containing gas and/or to the amount or fraction of oxygen within the oxygen-containing gas in real-time (or quasi-real time) during the delivery of the gas.

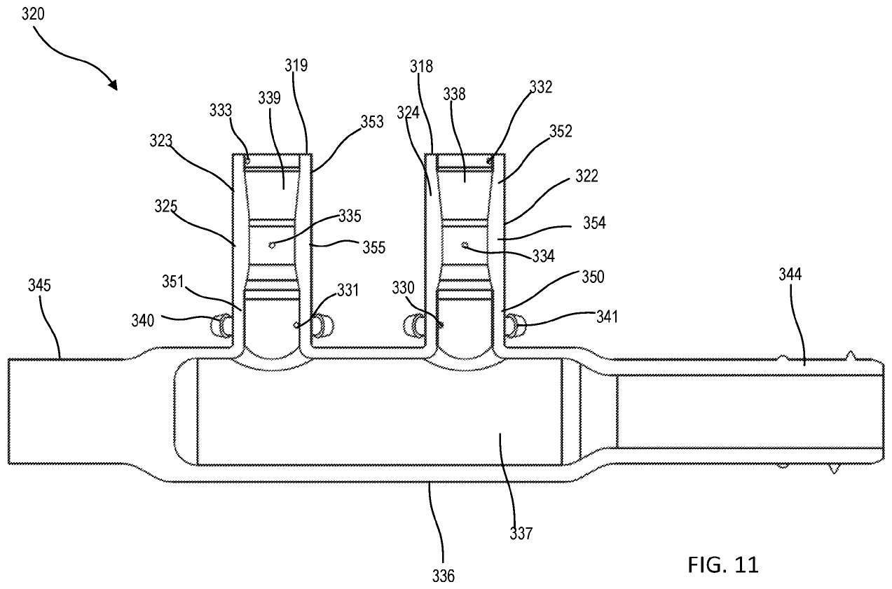

As shown in FIG. 11, the system 300 includes a nasal cannula 320, a pressure sensor (not shown) and a controller (not shown). The nasal cannula 320 includes a first nasal prong 322. a second nasal prong 323 and a base portion 336. The first nasal prong 322 includes a first end portion 350 that defines an inlet opening 326 and a second end portion 352 that defines an outlet opening 328, and a middle portion 354 between the first end portion 350 and the second end portion 352. A flow passage 338 is defined by an inner surface of a side wall 324 of the first nasal prong 322 between the inlet opening 326 and the outlet opening 328. The second nasal prong 323 includes a first end portion 351 that defines an inlet opening 327 and a second end portion 353 that defines an outlet opening 329, and a middle portion 355 between the first end portion 351 and the second end portion 353. A flow passage 339 is defined by an inner surface of a side wall 325 of the second nasal prong 323 between the inlet opening 327 and the outlet opening 329.

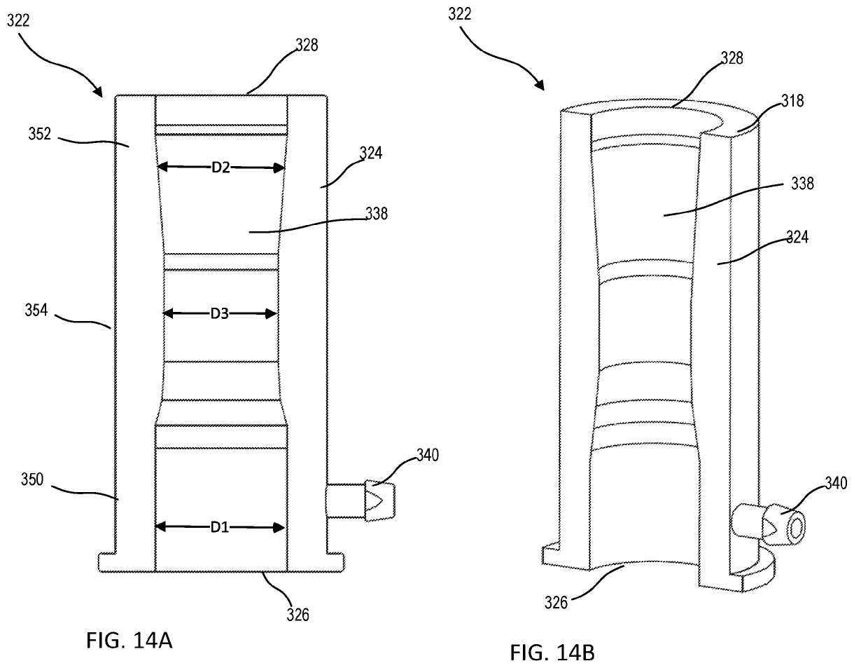

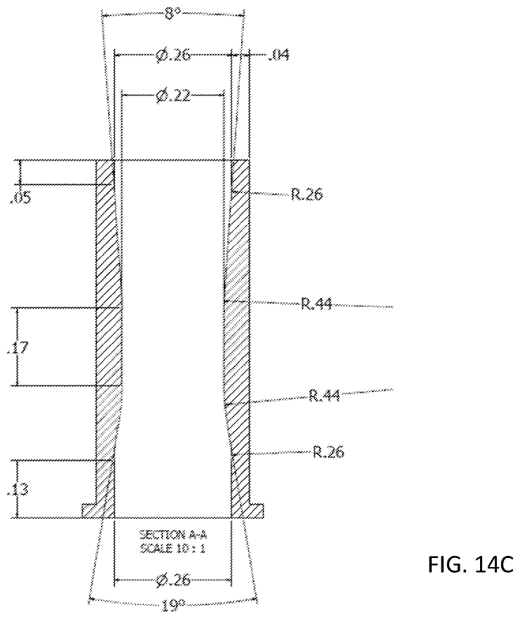

The side wall 324 of the first nasal prong 322 and the side wall 325 of the second nasal prong 323 can each have varying thickness such that an inner diameter of the flow passage 338 and an inner diameter of the flow passage 339 vary between the first end portions 350, 351 and the second end portions 352, 353. For example, as shown in FIG. 14A, the first nasal prong 322 can have a first inner diameter DI at the first end portion 350 and a second inner diameter D2 at the second end portion 352 that are each greater than an inner diameter D3 at the middle portion 354. The second nasal prong 323 can be similarly constructed. This construction provides for a venturi effect within the flow passages 338 and 339. FIG. 14C illustrates example inner diameters and wall thicknesses of the nasal prong 322 and example locations for the diameters and thicknesses.

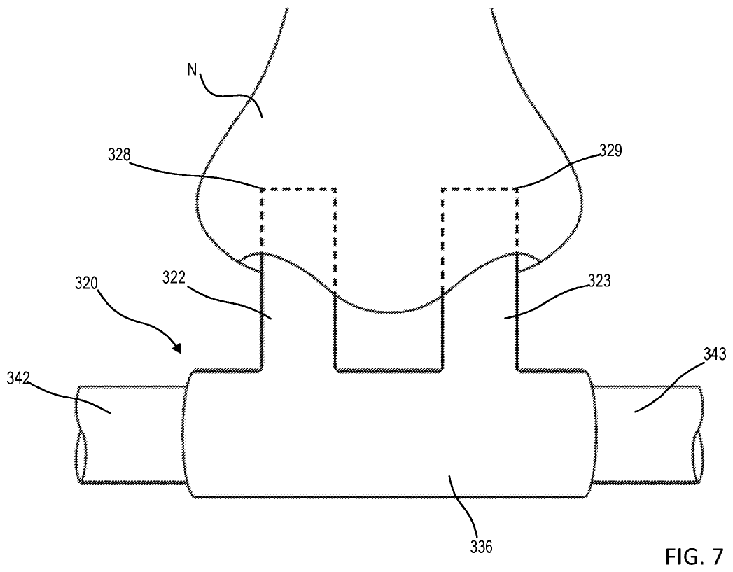

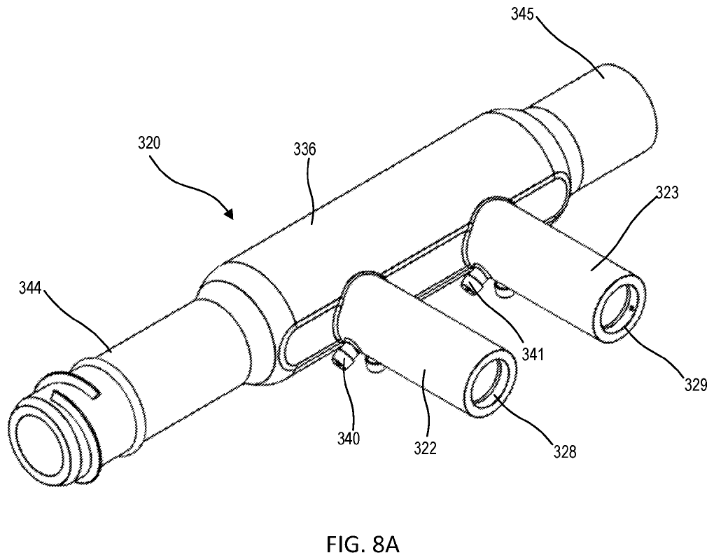

The first nasal prong 322 and the second nasal prong 323 are each configured to be inserted within a nostril of a patient such that the outlet opening 328 and the outlet opening 329 are each disposed within a different nostril of the nose N of a user, as shown for example, in FIG. 7. The nasal cannula 320 is couplable to a first support tube 342 and a second support tube 343. More specifically, the first support tube 342 is coupled to a first coupling portion 344 of the base portion 336 and the second support tube 343 is coupled to a second coupling portion 345 of the base portion 336. As shown, for example, in FIG. 6, the support tube 342 and the support tube 343 be used to support the nasal cannula 320 on the head of the patient by extending around the ears of the patient. The first support tube 342 and the second support tube 343 can also be coupled together at an adjustment member 321 that can be used to tighten or loosen the support tubes on the patient. The support tube 342 and/or the support tube 343 can be fluidically coupled to the source of oxygen-containing gas such that the gas can be delivered to the nasal cannula 320 via the first support tube 342 and/or the second support tube 343. For example, as shown in FIG. 11, the flow passage 338 can be in fluid communication with the first support tube 342 or the second support tube 343 via the interior region 337 of the base portion 336. In this embodiment, the oxygen-containing gas can flow through the first support tube 342, into the interior region 337 of the base portion 336 and into the first flow passage 338 and the second flow passage 339. The second coupling portion 345 of the base portion 336 is closed such that the gas does not flow past the first nasal prong 322 and the second nasal prong 323 and out through the second coupling portion 345. In other words, in this embodiment, the oxygen-containing gas only flows into the first support tube 342, and the second support tube 343 is only used to support the nasal cannula 320 on the user. In other embodiments, the oxygen-containing gas can flow into the nasal cannula 320 via the second support tube 343. In yet other embodiments, the oxygen-containing gas can flow into the nasal cannula 320 via each of the first support tube 342 and the second support tube 343.

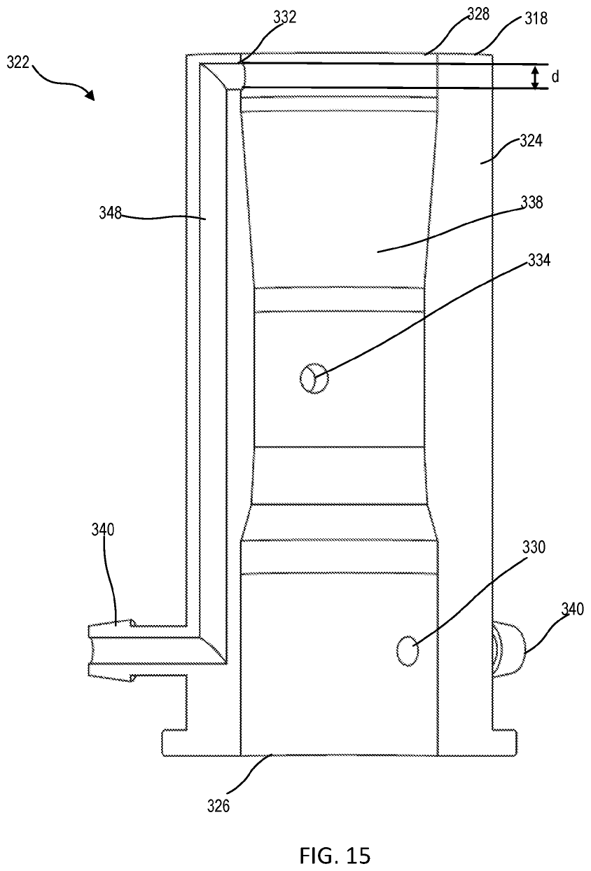

In this embodiment, each of the nasal prongs 322 and 323 have three ports that can be used to measure pressures within the flow passages 338 and 339. The side wall 324 of the first nasal prong 322 defines a first port 330 at a first location within the flow passage 338 at or near the first end portion 350, a second port 332 at a second location within the flow passage 338 at or near the second end portion 352, and a third port 334 at a third location within the flow passage 338 at or near the middle portion 354. Similarly, the side wall 325 of the second nasal prong 323 defines a first port 331 at a first location within the flow passage 339 at or near the first end portion 351, a second port 333 at a second location within the flow passage 339 at or near the second end portion 353, and a third port 335 at a third location within the flow passage 339 at or near the middle portion 355. As shown, for example, in FIG. 15, the second port 332 has a diameter d and is disposed at a distance from an end surface 318 of the first nasal prong 322 not more than two times the diameter of the second port 332. The second port 333 of the second nasal prong 323 can be similarly disposed near an end surface 319 of the second nasal prong 323. The close proximity of the ports 332 and 333 to the end surfaces 318 and 319 of the nasal prongs 322 and 323, respectively, provide for pressure measurements closer to the outlet openings of the nasal prongs 322 and 323, which can result in a more accurate determination of the inspiratory flow rate of the patient based on the pressure measurements, as described above with reference to the cannula 120.

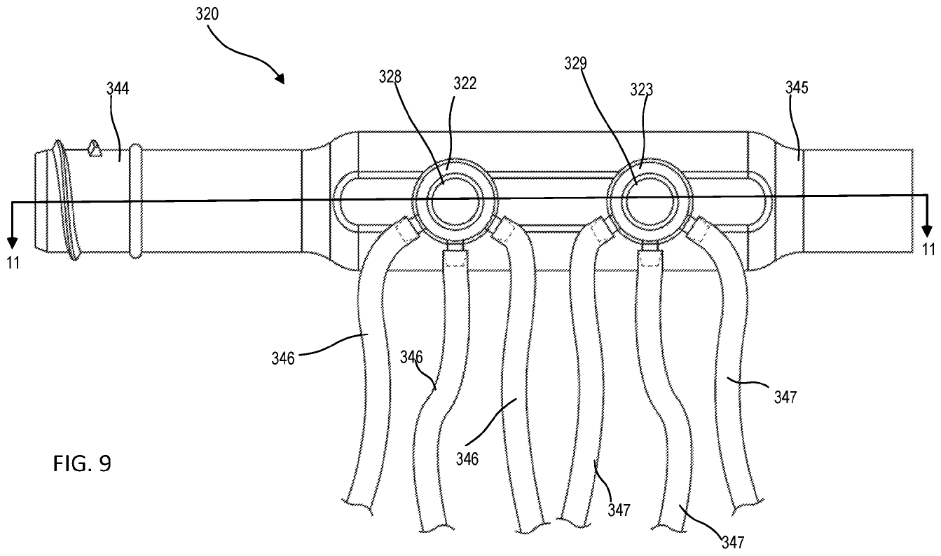

Each of the ports of the first nasal prong 322 and the second nasal prong 323 can be fluidically coupled to a pressure sensor (not shown in FIGS. 6-15) such that pressures within the flow passages 338 and 339 can be measured at each of the ports. For example, each of the ports 330, 332 and 334 of the first nasal prong 322 can be in fluid communication with separate pressure sensor couplers 340 disposed at the first end portion 350 of the nasal prong 322 via separate fluid port channels 348 defined in the side wall 324, as shown in FIG. 15 (only one fluid port channel 348 between the port 332 and its pressure sensor coupler 340 is shown). Separate pressure sensor tubes 346 can be coupled to and in fluid communication with the pressure sensor couplers 340, as shown in FIG. 9. Similarly, each of the ports 331, 333 and 335 of the second nasal prong 323 can be in fluid communication with a separate pressure sensor coupler 341 disposed at the first end portion 351 of the nasal prong 323 via a fluid port channel (not shown). Separate pressure sensor tubes 347 can be coupled to and in fluid communication with the pressure sensor couplers 341, as shown in FIG. 9. Although not shown, in some embodiments, the pressure sensor tubes 346 and 347 can be gathered and coupled together for ease of use on the patient. In some embodiments, the pressure sensor tubes can be coupled to (or constructed together with) the first support tube 342 or the support tube 343.

The pressure sensor tubes 346 and 347 can each be coupled to a pressure sensor and used to measure pressures within the flow passage 338 of the nasal prong 322 and the flow passage 339 of the nasal prong 323. As described above for previous embodiments, pressure measurements can be measured within the flow passages 338 and 339 during delivery of the oxygen-containing gas to the nasal cannula 320 and used to determine an inspiratory flow rate of the patient.

As described above for previous embodiments, a controller (not shown, but which can be similar to any of the controllers described herein) can have a processor (not shown) operatively coupled to the pressure sensors. The processor is configured to produce one or more pressure waveforms characterizing the pressure measurements at each location as a function of time during the delivery of the oxygen-containing gas to the patient in the same manner as described above. In some embodiments, the processor can calculate a difference between the pressure measurements associated with the different ports of the first nasal prong 322 and a difference between the pressure measurements associated with the different ports of the second nasal prong 323. Based on the pressure differences (e.g., at the throat of the venturi and at the first end portion of the prongs) the flow rate of the oxygen-containing gas through each nasal prong can be estimated. Additionally, based on at least any of the pressure measurements (or the pressure waveforms produced therefrom), the processor can determine any of an inspiratory flow rate of the patient, a tidal volume of the patient, or a minute ventilation of the patient during the delivery of the oxygen-containing gas. The processor can produce an output based at least in part on the determined inspiratory flow rate, tidal volume, or minute ventilation. For example, the output produced by the processor can include a control signal that causes an adjustment to at least one of a percentage of oxygen within the oxygen-containing gas or a flow rate of the oxygen-containing gas being delivered into the nasal cannula 320 based at least in part on the determined inspiratory flow rate. In some embodiments, the control signal can be sent to a flow control valve (not shown) that is coupled to the source of the oxygen-containing gas and coupled to the nasal cannula 320 to deliver the oxygen-containing gas to the nasal cavity of the patient. With information related to the patient's inspiratory flow rate determined by the processor, adjustments can automatically be made to the flow of the oxygen-containing gas in real-time during the delivery of the oxygen-containing gas. As also described above for previous embodiments, the processor can receive input including data related to a health parameter associated with the patient. In such a case, the processor can produce the control signal based at least in part on the determined inspiratory flow rate and the health parameter(s).

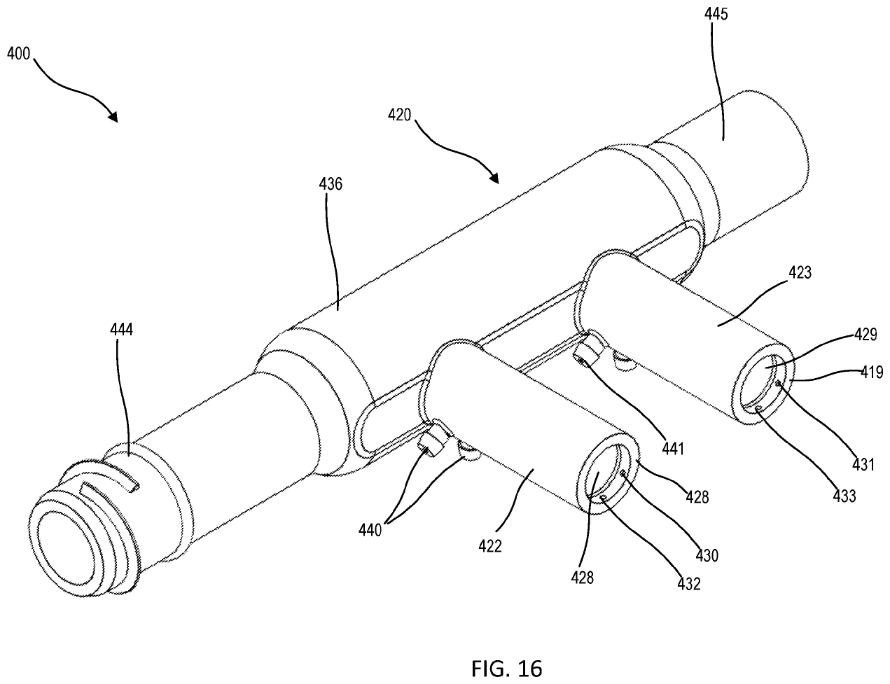

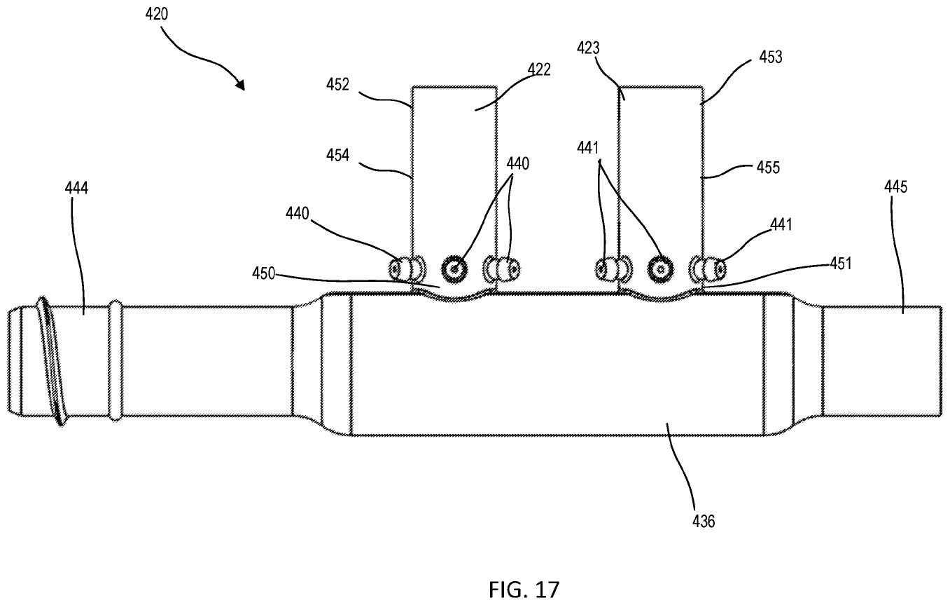

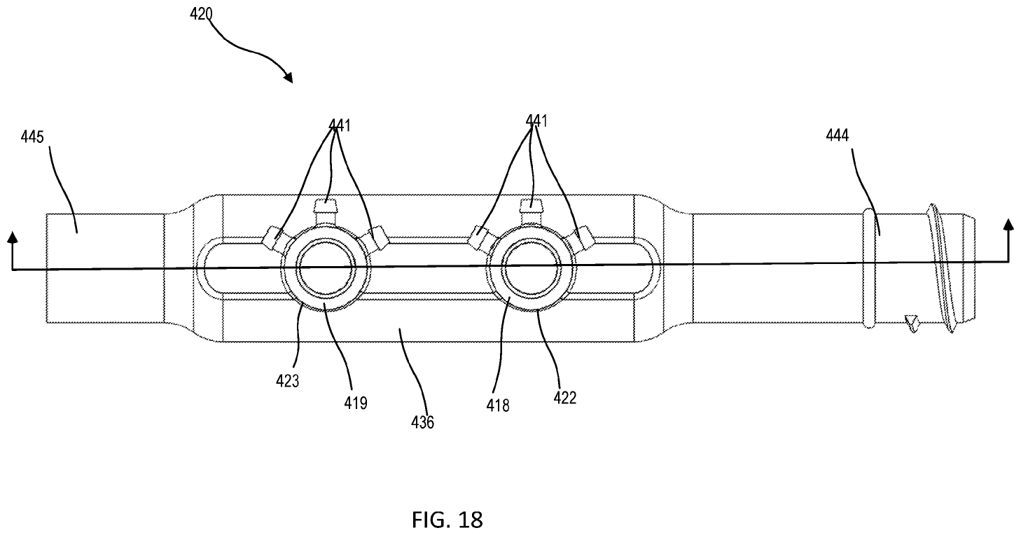

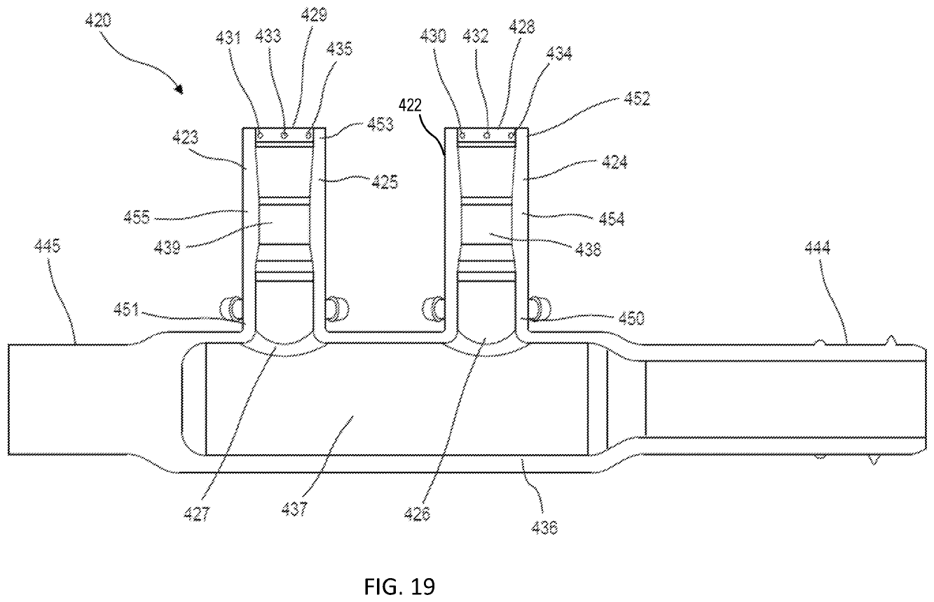

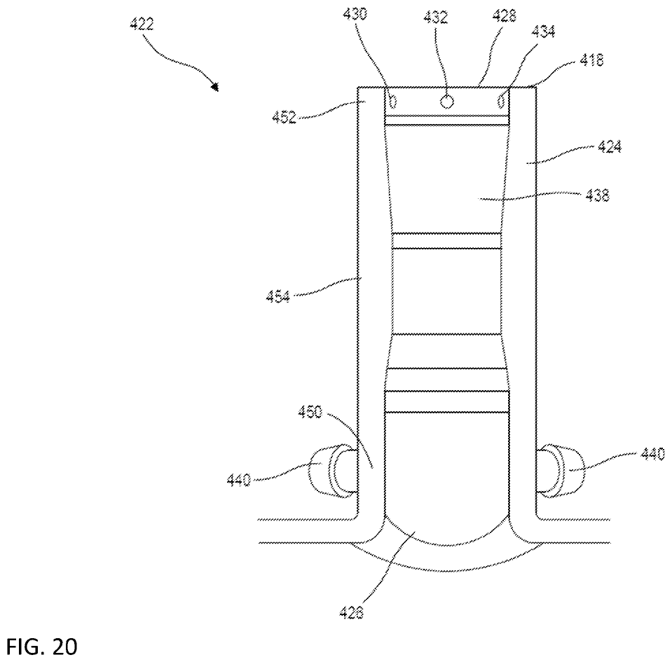

FIGS. 16-21 illustrate portions of a flow regulated nasal delivery system (also referred to as “flow regulated system” or “delivery system” or “system”), according to another embodiment. The system 400 can be used to deliver an oxygen-containing gas to a patient and provide real-time information related to the patient's inspiratory flow rate during the delivery of the gas and can include the same or similar features and function the same or similar as the systems 100 and 300 described above. Thus, some features are not shown and described with reference to this embodiment. As with the previous embodiment, the system can make automatic adjustments to the flow rate of the oxygen-containing gas and/or to the amount or fraction of oxygen within the oxygen-containing gas in real-time during the delivery of the gas.

The system 400 includes a nasal cannula 420, a pressure sensor (not shown) and a controller (not shown). The nasal cannula 420 includes a first nasal prong 422, a second nasal prong 423 and a base portion 436. The first nasal prong 422 includes a first end portion 450 that defines an inlet opening 426 and a second end portion 452 that defines an outlet opening 428, and a middle portion 454 between the first end portion 450 and the second end portion 452. A flow passage 438 is defined by an inner surface of a side wall 424 of the first nasal prong 422 between the inlet opening 426 and the outlet opening 428. The second nasal prong 423 includes a first end portion 451 that defines an inlet opening 427 and a second end portion 453 that defines an outlet opening 429, and a middle portion 455 between the first end portion 451 and the second end portion 453. A flow passage 439 is defined by an inner surface of a side wall 425 of the second nasal prong 423 between the inlet opening 427 and the outlet opening 429.

The side wall 424 of the first nasal prong 422 and the side wall 425 of the second nasal prong 423 can each have varying thickness such that an inner diameter of the flow passage 438 and an inner diameter of the flow passage 439 vary between the first end portions 450, 451 and the second end portions 452, 453 in the same or similar manner as described for cannula 320. This construction provides for a venturi effect within the flow passages 438 and 439 (which can enable determination of the flow rate of gas through each flow passage). As described above, FIG. 14C illustrates example inner diameters and wall thicknesses of a nasal prong and example locations for the diameters and thicknesses that can apply to cannula 420.

The first nasal prong 422 and the second nasal prong 423 are each configured to be inserted within a nostril of a patient such that the outlet opening 428 and the outlet opening 429 are each disposed within a different nostril of the nose of a user, in the same manner as cannula 320 as shown for example, in FIG. 7. Although not shown, the nasal cannula 420 is couplable to a first support tube and a second support tube as shown for cannula 320 and support tubes 342 and 343. More specifically, a first support tube can be coupled to a coupling portion 444 of the base portion 436 and a second support tube can be coupled to a coupling portion 445 of the base portion 436. The support tubes can be used to support the nasal cannula 420 on the head of the patient by extending around the ears of the patient. The first support tube and the second support tube can also be coupled together at an adjustment member that can be used to tighten or loosen the support tubes on the patient. At least one of the support tubes can be fluidically coupled to the source of oxygen-containing gas such that the gas can be delivered to the nasal cannula 420 via the first support tube and/or the second support tube. For example, the flow passage 438 can be in fluid communication with the first support tube or the second support tube via the interior region 437 of the base portion 436. In this embodiment, the oxygen-containing gas can flow through the first support tube, into the interior region 437 of the base portion 436 and into the first flow passage 438 and the second flow passage 439. As with cannula 320, the second coupling portion 445 of the base portion 436 is closed such that the gas does not flow past the first nasal prong 422 and the second nasal prong 423 and out through the second coupling portion 445. In other words, in this embodiment, the oxygen-containing gas only flows into the first support tube, and the second support tube is only used to support the nasal cannula 420 on the user.

Each of the nasal prongs 422 and 423 have three ports that can be used to measure pressures within the flow passages 438 and 439. In this embodiment, each of the ports are defined at the second end portion of the respective nasal prong. More specifically, the side wall 424 of the first nasal prong 422 defines a first port 430 at a first radial location within the flow passage 438 at or near the second end portion 452, a second port 432 at a second radial location within the flow passage 438 at or near the second end portion 452, and a third port 434 at a third radial location within the flow passage 438 at or near the second end portion 452. Similarly, the side wall 425 of the second nasal prong 423 defines a first port 431 at a first radial location within the flow passage 439 at or near the second end portion 453, a second port 433 at a second radial location within the flow passage 439 at or near the second end portion 453, and a third port 435 at a third radial location within the flow passage 439 at or near the second end portion 455. In some embodiments, each of the three ports of the first nasal prong 422 are disposed at a distance from an end surface 418 of the first nasal prong 422 not more than two times a diameter of each (or any) of the three ports. In other embodiments, each of the three ports of the first nasal prong 422 are disposed at a distance from an end surface 418 of the first nasal prong 422 not more than four times a diameter of each (or any) of the three ports. Similarly, in some embodiments, each of the three ports of the second nasal prong 423 are disposed at a distance from an end surface 419 of the second nasal prong 423 not more than two times a diameter of the three ports. In some embodiments, each of the three ports of the second nasal prong 423 are disposed at a distance from an end surface 419 of the second nasal prong 423 not more than four times a diameter of each (or any) of the three ports. The close proximity of the ports to the end surfaces 418 and 419 of the nasal prongs 422 and 423, respectively, provide for pressure measurements closer to the outlet openings of the nasal prongs 422 and 423, which can result in a more accurate determination of the inspiratory flow rate of the patient based on the pressure measurements. The inclusion of multiple ports at each longitudinal location within the nasal prongs provides for redundancy of measurement of the pressure.

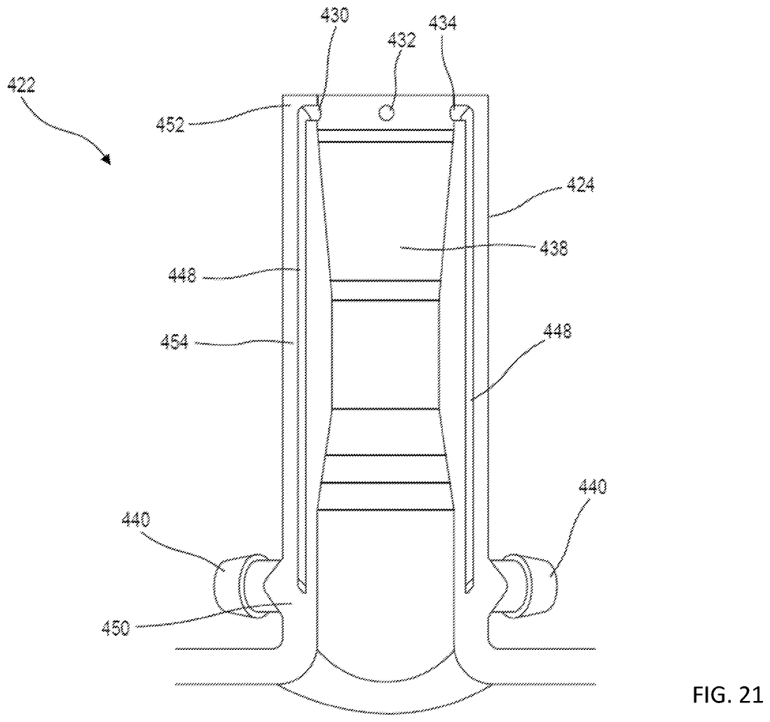

Each of the ports of the first nasal prong 422 and the second nasal prong 423 can be fluidically coupled to a pressure sensor (not shown in FIGS. 16-21) such that pressures within the flow passages 438 and 439 can be measured at the ports. For example, each of the ports 430, 432 and 434 of the first nasal prong 422 can be in fluid communication with a separate pressure sensor coupler 440 disposed at the first end portion 450 of the nasal prong 422 via a fluid port channel 448 defined in the side wall 424, as shown in FIG. 21. Separate pressure sensor tubes (not shown) can be coupled to and in fluid communication with the pressure sensor couplers 440, in a similar manner as described above and as shown in FIG. 9. Similarly, each of the ports 431, 433 and 435 of the second nasal prong 423 can be in fluid communication with a separate pressure sensor coupler 441 disposed at the first end portion 451 of the nasal prong 423 via a fluid port channel (not shown). Separate pressure sensor tubes (not shown) can be coupled to and in fluid communication with the pressure sensor couplers 441, as described above and shown in FIG. 9. Although not shown, in some embodiments, the pressure sensor tubes can be gathered and coupled together for ease of use on the patient.

The pressure sensor tubes can each be coupled to a pressure sensor and used to measure pressures within the flow passage 438 of the nasal prong 422 and the flow passage 439 of the nasal prong 423. As described above for previous embodiments, pressure measurements can be measured within the flow passages 438 and 439 during delivery of the oxygen-containing gas to the nasal cannula 420 and used to determine an inspiratory flow rate of the patient.

As described above for previous embodiments, a controller (not shown, but which can be similar to any of the controllers described herein) can have a processor (not shown) operatively coupled to the pressure sensors. The processor is configured to produce one or more pressure waveforms characterizing the pressure measurements at each location as a function of time during the delivery of the oxygen-containing gas to the patient in the same manner as described above. Based on at least any of the pressure measurements (or the pressure waveforms produced therefrom), the processor can determine any of an inspiratory flow rate of the patient, a tidal volume of the patient, or a minute ventilation of the patient during the delivery of the oxygen-containing gas. The processor can produce an output based at least in part on the determined inspiratory flow rate, tidal volume, or minute ventilation. For example, the output produced by the processor can include a control signal that causes an adjustment to at least one of a percentage of oxygen within the oxygen-containing gas or a flow rate of the oxygen-containing gas being delivered into the nasal cannula 420 based at least in part on the determined inspiratory flow rate. In some embodiments, the control signal can be sent to a flow control valve (not shown) that is coupled to the source of the oxygen-containing gas and coupled to the nasal cannula 420 to deliver the oxygen-containing gas to the nasal cavity of the patient. With information related to the patient's inspiratory flow rate determined by the processor, adjustments can automatically be made to the flow of the oxygen-containing gas in real-time during the delivery of the oxygen-containing gas. As also described above for previous embodiments, the processor can receive input including data related to a health parameter associated with the patient. In such a case, the processor can produce the control signal based at least in part on the determined inspiratory flow rate and the health parameter(s).

As shown in FIG. 22, a schematic diagram of one embodiment of suitable components that may be included within the controller (e.g., 105) of any of the systems described herein is illustrated. In some embodiments, and as described above, the controller 105 can include one or more processor(s) 110 and associated memory device(s) 111 configured to perform a variety of computer implemented functions (e.g., performing the methods, steps, calculations and the like and storing relevant data as disclosed herein). Additionally, in some embodiments, the controller 105 includes a communication module 112 to facilitate communications between the controller 105 and the various components of the system 100.

As used herein, the term “processor” refers not only to integrated circuits referred to in the art as being included in a computer, but also refers to a controller. a microcontroller, a microcomputer, a programmable logic controller (PLC), an application specific integrated circuit, and other programmable circuits. Additionally, the memory device(s) 111 may generally comprise memory element(s) including, but not limited to, computer readable medium (e.g., random access memory (RAM)), computer readable nonvolatile medium (e.g., a flash memory), a floppy disk, a compact disc read only memory (CD ROM), a magneto optical disk (MOD), a digital versatile disc (DVD) and/or other suitable memory elements. Such memory device(s) 111 may generally be configured to store suitable computer readable instructions that, when implemented by the processor(s) 110, configure the controller 105 to perform various functions.

The communication module 112 may include a control input module 113, a pressure sensor interface module 114, a flow rate module 116, and a device control module 117. The control input module 113 can be configured to receive control inputs from the operator/care giver. The pressure sensor interface module 114 (e.g., one or more analog to digital converters) can be configured to permit signals transmitted from one or more pressure sensors (e.g., pressure sensor 115 of the system 100) to be converted into signals that can be understood and processed by the processors 110. The pressure sensors may be communicatively coupled to the communication module 112 using any suitable means. For example, the pressure sensors may be coupled to the communication module 112 via a wired connection and/or via a wireless connection, such as by using any suitable wireless communications protocol known in the art. The flow rate module 116 can be used to determine the inspiratory flow rate of a patient as described herein. The device control module 117 can be configured to communicate with a source of oxygen-containing gas or a control valve as described herein to adjust a flow rate of the oxygen-containing gas being delivered to a nasal cannula (e.g., nasal cannula 120) or adjust the amount of oxygen within the oxygen-containing gas being delivered to a patient.

As described above, any of the systems and apparatus described herein can be used in connections with multiple different use modes, including (but not limited to) an autonomous mode, a manual mode and a code mode. The systems described herein are designed to optimize oxygen delivery, reduce dead space, improve mechanics, and reduce work of breathing by the patient. By determining trends in tidal volume, minute ventilation, and/or inspiratory flow rates, early predictions of success or failure of noninvasive ventilation can be assessed. Any of the systems described herein can operate in an autonomous mode when oxygen saturation wave forms are validated, making it less labor intensive for the care giver. In contrast, manual or semiautonomous modes can be utilized in situations where oxygen saturation wave forms are poor or where oxygen saturation monitoring is not possible.

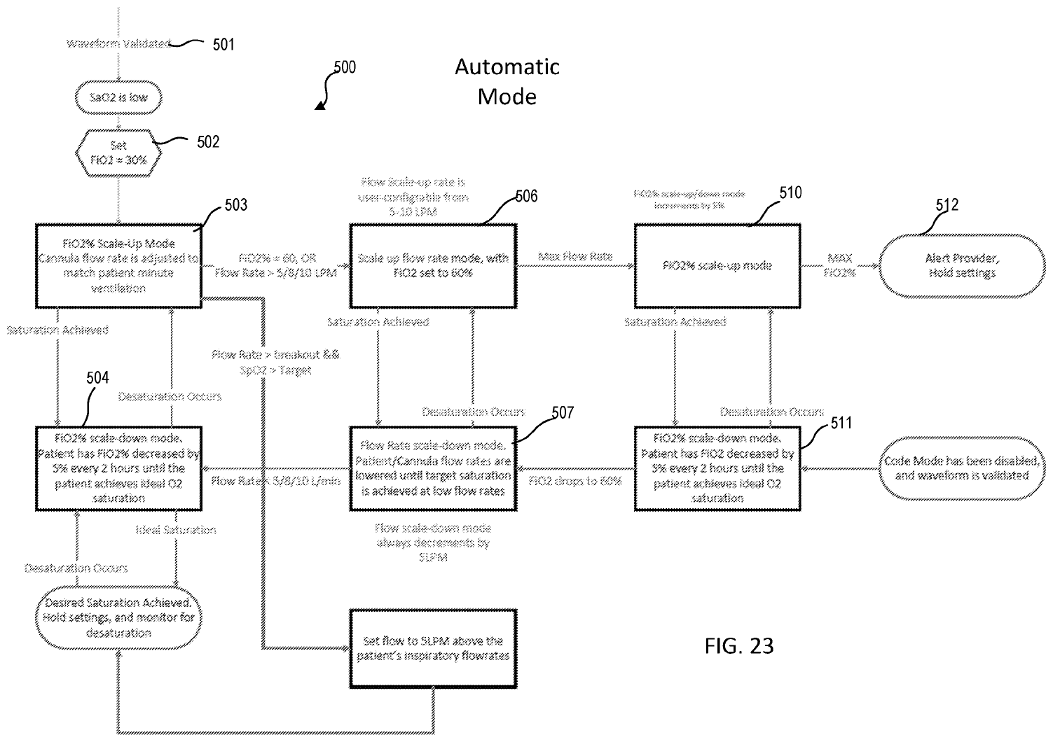

FIGS. 23-25 are flow charts illustrating examples of additional methods that can be performed during operation of the systems described herein (including any of the nasal cannulas, controllers, or other aspects of any of the systems described herein). FIG. 23 shows a method of operation 500 within an autonomous (or fully automatic) mode. The automatic mode uses both measurements of the patient's oxygen saturation (indicated as SaO2 in FIG. 23 and the description below) and the determined inspiratory flow rate (based on the cannula pressure measurements) to control the amount of oxygen delivered to the patient. Similarly stated, the system can receive input related to a health parameter associated with the patient (e.g., the SaO2) and also the pressure measurements from the cannula and employ a cascaded control method to accurately control the amount of oxygen delivered to the patient. As described below, the automatic mode method controls the amount of oxygen delivered based on the patient's inspiratory flow rate as a first (or an inner) control loop to effectively match the flow rate of oxygen-containing gas with the patient's inspiratory flow rate, while also including a second (or an outer) control loop based on the measured oxygen saturation. As shown in FIG. 23, the controller initially receives a waveform or other information associated with the patient's oxygen saturation (SaO2). The SaO2 waveform and/or information can be generated by any suitable means (either within the system or from an external system), such as for example via a pulse oximetry system. The controller (or processor) validates the SaO2 waveform or information, at 501. If the SaO2 information is determined to be unreliable, the system will exit the automatic mode and produce a suitable notification for the user. If the SaO2 is validated and determined to be lower than the desired set point, the concentration of the oxygen within the oxygen-containing gas (referred to as the fraction of inspired oxygen, FiO2) is set to an initial setpoint, at 502. Although the initial setpoint is shown as being 30%, in other embodiments, the initial setpoint for FiO2 can be any suitable value (e.g., 32%, 34%, 36%, or 38%). The method then enters into a FiO2 control algorithm, during which the FiO2 will be increased (or decreased) to achieve the desired SaO2 setpoint. This is identified in FIG. 23 as the FiO2 “scale-up” (operation 503) or “scale-down” mode (operation 504). During the FiO2 control algorithm. the flow rate of the oxygen-containing gas is also adjusted based on any of the inspiratory flow rate, tidal volume, or minute ventilation as determined based on the cannula pressure measurements. Similarly stated, the flow rate of the oxygen-containing gas is matched to the patient's inspiratory flow rates. Thus, the method employs multiple control loops to improve accuracy of the amount of oxygen delivered.

Specifically, at operation 503, the system determines any of the patient's inspiratory flow rate, tidal volume, or minute ventilation using any of the systems described herein and according to any of the methods described herein. The flow rate of the oxygen-containing gas is adjusted (e.g., via sending one or more control signals, as described herein) to match the gas flow rate to the patient's inspiratory flow rate and/or minute ventilation. So, in this example, the initial gas flow to the patient would be delivered at a flow rate that is associated with the patient's inspiratory flow rate and with an oxygen concentration of 30%. When the SaO2 input is less than the desired oxygen saturation. the system increases FiO2 at a desired increments while the gas flow rate is also being adjusted to match the patient's inspiratory flow rate. In some embodiments, the FiO2 can be increased in increments of 5% over a series of two-minute intervals until the desired saturation is achieved. At this point the system holds the FiO2 and continues matching the patient's inspiratory flows. In some embodiments, when the desired oxygen saturation is achieved, the method can include a “hold mode” that keeps the gas flow matched (or held constant) and FiO2 at a steady state level for two hours. After two hours, the system attempts to decrease the FiO2, at 504. If no oxygen desaturation is detected, the system would go into a new holding and monitoring phase for another two hours and repeat the process. In other embodiments, the time intervals and increments of changing the FiO2 can be any suitable values.

In some embodiments, the method can include exiting the FiO2 control algorithm portion in response to reaching one or more control limits associated with any combination of the FiO2, the patient's inspiratory flow rate, or the flow rate of the oxygen-containing gas. For example, in some embodiments, the method can exit the FiO2 control algorithm when each of (a) the gas flow rate is greater than or equal to, for example, 5, 8, or 10 L/min, and (b) FiO2 of 60% is reached without achieving desired saturation. In some embodiments, the method can exit the FiO2 control algorithm when each of (a) the gas flow rate is greater than or equal to 15 L/min, and (b) FiO2 of 60% is reached without achieving desired saturation. Reaching these two conditions without achieving the desired oxygen saturation causes the system to enter a gas flow rate scale up mode, at 506. In this mode, the system will increase the flow rate of the oxygen-containing gas at a predetermined increment (e.g., 5 L/min) for a desired increment (e.g., every two minutes) while holding FiO2 at 60% until desired oxygen saturation is achieved or the system reaches its maximum flow capacity.

In some embodiments, when the desired oxygen saturation is achieved, the method can include a flow rate scale down mode, at 507 that keeps the gas flow held constant and FiO2 at a steady state level (e.g., 60%) for two hours. The system decreases the oxygen-containing gas flow rate until a target saturation is achieved at low flow rates. For example, in some embodiments, after two hours, the system decreases the oxygen-containing gas flow rate by 5 L/min. If no oxygen desaturation is detected, the system would go into a new holding and monitoring phase for another two hours and repeat the process. In this example, if the gas flow rate drops to below a predetermined value (e.g., below 5, 8, 10 or 15 L/min), the method can include re-entering to the FiO2 control algorithm at operations 503 or 504 as discussed above. In other embodiments, the time intervals and increments of changing the gas flow rate can be any suitable values.

In some embodiments, the method will include causing the system to enter a second FiO2 scale-up mode at 510 if the desired saturation is not achieved at a maximum flow rate and FiO2 of 60%. In the second FiO2 scale-up mode, the system starts ramping up FiO2 by a predetermined increment (e.g., 5%) over a predetermined time interval (e.g., two minute intervals) until the desired SaO2 is achieved. In this mode, the oxygen concentration will be increased beyond the 60% FiO2 value that was maintained constant during operations 506 and 507. Once the desired SaO2 is achieved, the system holds the flows and FiO2 in a steady state for two hours, at 511. If SaO2 is achieved, or is greater than desired, the system can then start to decrease FiO2 by a predetermined increment (e.g., 5%) over a predetermined time interval (e.g., two hours), at 511. Once FiO2 is decreased to 60%. the system returns to the flow control algorithm at 506 to titrate the flow down based on previously described process until the gas (or inspiratory flow) is less than or equal to 15 L/min. The system at this point starts to titrate FiO2 further down following previous cyclic time intervals in operations 503 and 504 to the minimum FiO2 needed to maintain desired SaO2 (in this example, the minimum Fio2 can be 30% or more). At any point during this process, if patient desaturates the system can return to the second FiO2 scale-up mode (e.g., increase the FiO2 by 5% every 2 minutes until desired saturation is achieved).

If desired saturation is not achieved at maximum flow and maximum FiO2, the system would alarm and notify the provider, at 512. If during the autonomous mode, SaO2 wave form is lost or becomes uninterpretable, the system would provide the most recent FiO2 and gas flow rates that were achieved in the autonomous mode and alert the provider. If SaO2 is validated again or the signal is regained, the system would start from the previous flows and FiO2 values.

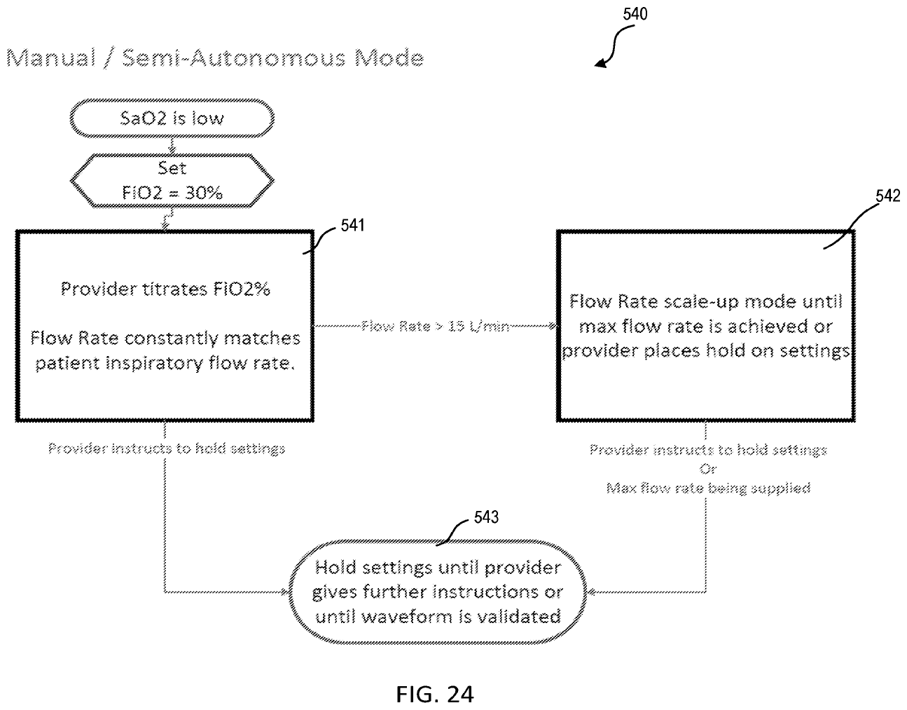

FIG. 24 shows a method of operation 540 within a manual/semi-autonomous mode. The semi-automatic mode can be used to allow a user to manually titrate the oxygen, for example, in situations where the SaO2 waveform or information is not validated. Thus, the semi-automatic mode uses measurements of the determined inspiratory flow rate (based on the cannula pressure measurements) to control the amount of oxygen delivered to the patient. In this case, the system provides the flow which is equal to the patient's flow and FiO2 is titrated by the provider, at 541. In semi-autonomous mode, when the system senses the gas flow (or patient's inspiratory flow rate) being greater than 15 L/min, the method includes activating a scale up mode at 541 where the system starts to ramp up flows by a predetermined flow increment (e.g., 5 L/min) over a predetermined time interval (e.g., two minutes). This occurs until a maximum flow rate is achieved or until the provider places the system in a hold mode (At 543) at particular flow rate.

In the autonomous and semi-autonomous modes, the system can also account for certain health conditions and events of the patient, such as a cough, sneeze and sigh. For example, the system can recognize sudden massive increases in flows during a cough, sneeze, and a sigh. The system can then provide matching flows during these events; however, it may not increase the flows permanently. This is achieved by the system validating the flows for short time intervals (e.g., a 30 second period) before permanently increasing to a new rate of flow delivery.

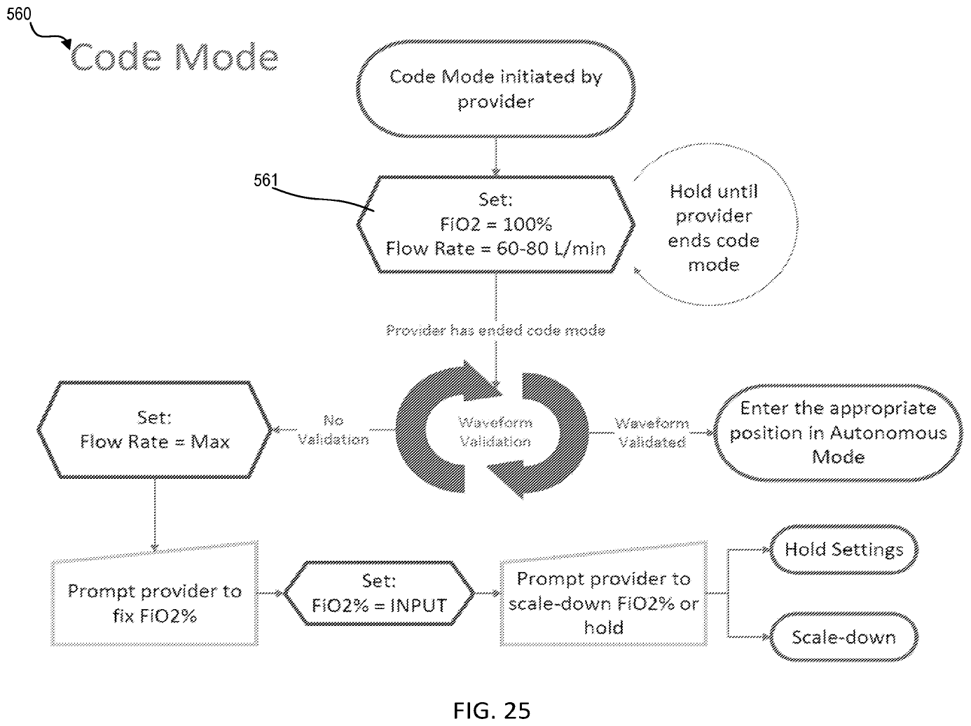

FIG. 25 shows a method of operation 560 within a code mode, which can be used when a patient is in severe distress or in a hospital code situation (e.g., a cardiac arrest code or the like). When the system is placed in the code mode. the method includes delivering oxygen-containing gas at 100% FiO2 at a high flow rate (e.g., 60-80 L/min), at 561. Once the system authenticates the oxygen saturation wave form, the user can then enter an input to place the system into autonomous (or semi-autonomous) mode and hold the code setting for predetermined time period (e.g., two hours). Next, the system would start to titrate FiO2 down according to the previously described process until FiO2 reaches 60%. Next the system would start reducing the flow rate of gas according to the previously described process, monitoring and maintaining the oxygen saturation levels. Flows would continue to be titrated down at previously described intervals until flows match the patient's inspiratory flows. At this time, the system would start titrating down the FiO2 according to the previously described intervals.

### Experimental Results

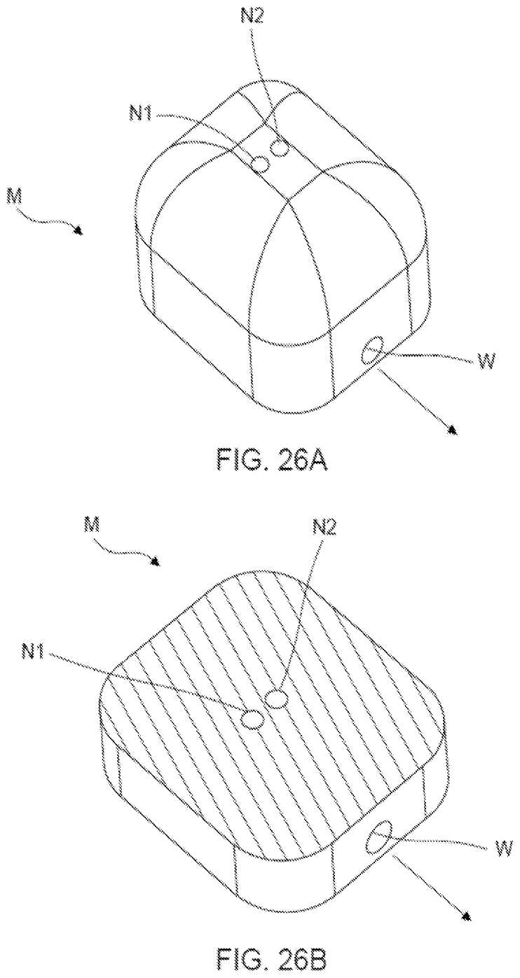

A series of bench tests were performed to produce an initial correlation of nasal cannula pressure to the inspiratory flow rate. The bench tests were performed using a system similar to the system 300 and a nasal cannula similar to the nasal cannula 320 described herein. Referring to FIGS. 26A and 26B, the test included inserting the nasal prongs (e.g., prongs 322, 323) into a physical model that simulates two nostrils (N1 and N2) and a windpipe W that connects the nostrils to a simulated lung (not shown). The simulated lung is a positive displacement syringe pump that can be actuated to control (and measure) the volume of air inhaled and exhaled through the nostrils. The model M does not include a mouth or other exit point. thereby ensuring that all of the measured volume inhaled and exhaled flows through the nostrils N1 and N2. During the tests, the nasal cannula (not shown in FIGS. 26A and 26B) was connected to a source of gas via a supply tube (similar to the support tube 343 described above). Thus, during the tests, the gas flow (similar to the gas flow Fc as shown in FIGS. 2A and 2B) could be carefully controlled. Tests were run with gas flows ranging from zero to 40 L/min (20 L/min per nasal prong). Tests were also conducted by varying the amount of inhalation and exhalation volume to simulate short, shallow breaths and long, deep breaths. Pressure was measured at all cannula pressure ports, and the data presented here is for pressure measured at the cannula tip (i.e., at a location similar to that of the ports 332 and 333 described above).

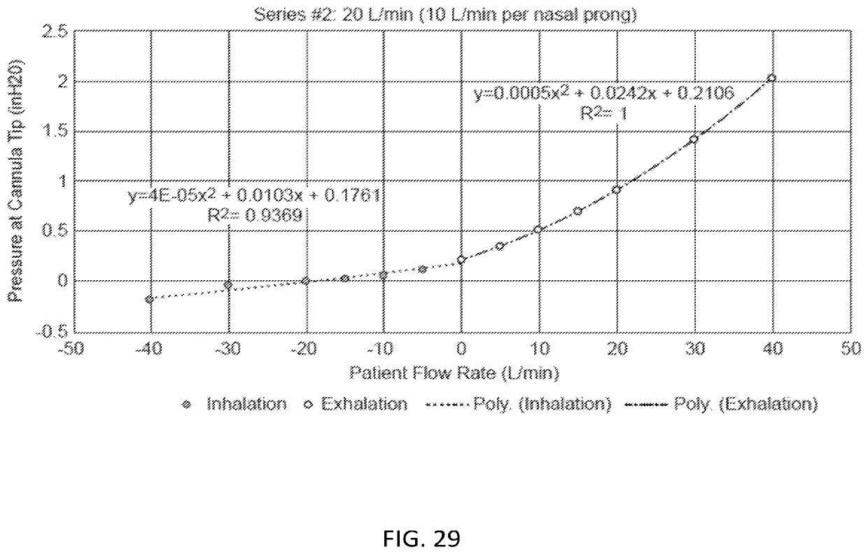

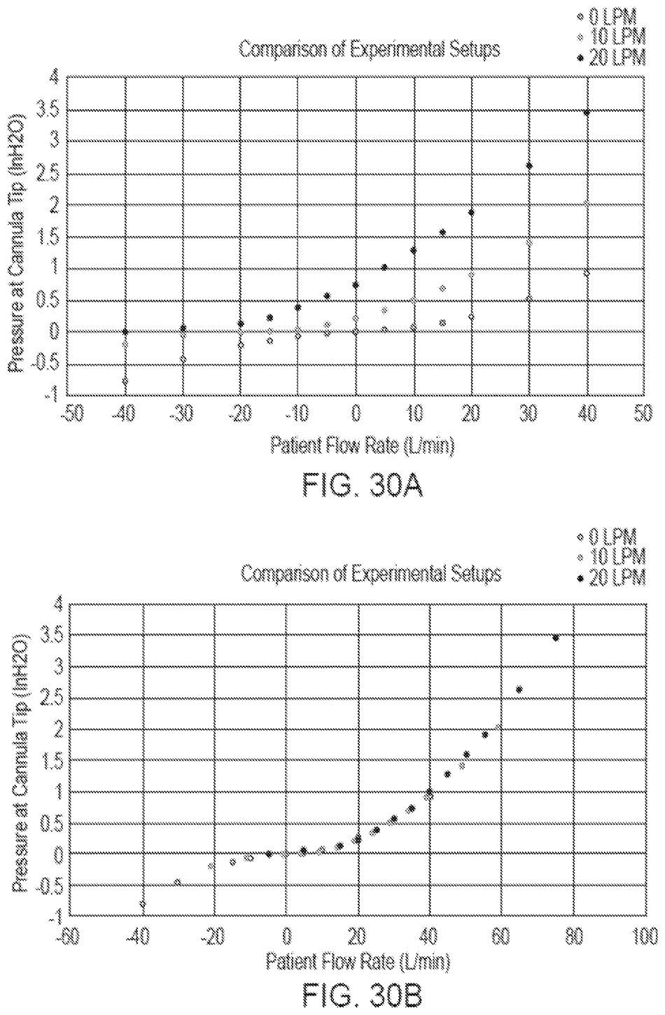

FIG. 27A is a plot of the cannula tip pressure as a function of time measured during a bench test with total cannula gas flow rate of 20 L/min (i.e., roughly 10 L/min per nasal prong) and inspiration volume of 500 mL. The left and right markers identify the beginning and ending of each exhalation event and inhalation event. As shown, the cannula tip pressure is positive during the exhalation and is slightly negative during the inhalation. FIG. 27B is a plot of the calculated inspiratory flow rate as a function of time based on the pressure waveform. The calculated inspiratory flow rate is based on the numerical modeling discussed below with reference to FIGS. 29, 30A and 30B.

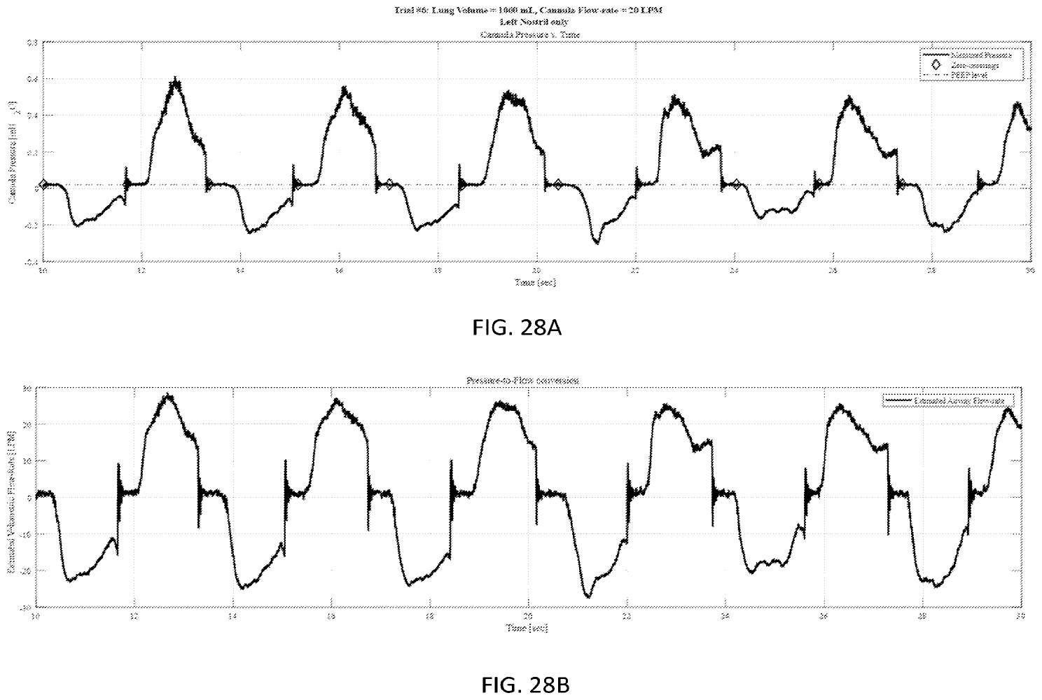

FIG. 28A is a plot of the cannula tip pressure as a function of time measured during a bench test with total cannula gas flow rate of 20 L/min (i.e., 10 L/min per nasal prong) and inspiration volume of 1000 mL. The left and right markers identify the beginning and ending of each exhalation event and inhalation event. As shown, the cannula tip pressure is positive during the exhalation and is negative during the inhalation. Because the amount of inspiration volume is higher (by a factor of two), the magnitude of the cannula tip pressure is higher than that presented in FIG. 27A. FIG. 28B is a plot of the calculated inspiratory flow rate as a function of time based on the pressure waveform. The calculated inspiratory flow rate is based on the numerical modeling discussed below with reference to FIGS. 29, 30A and 30B.

A calibration curve was developed for each series of tests run at a constant gas flow rate. For example, FIG. 29 is a plot of the cannula tip pressure as a function of the patent flow rate as measured during a series of bench tests where the total cannula gas flow rate is 20 L/min (i.e., roughly 10 L/min for each prong). In these tests, the total amount of flow is the independent variable and is controlled by the positive displacement syringe (which simulates the lungs). The calibration can be generated by maintaining a steady flow rate of inhalation and measuring the pressure during this steady state condition. As shown in FIG. 29, there is a second order correlation between the inspiratory flow rate and the measured pressure. Specifically, there are separate calibration curves for the inspiration and expiration cycles.

FIG. 30A is a plot showing the measured cannula tip pressure as a function of the inspiratory flow rate from a series of bench tests with varying nasal prong gas flow rate. As shown, the higher gas flow rate results in higher cannula tip pressure. FIG. 30B is a plot showing the measured cannula tip pressure as a function of the inspiratory flow rate with the flow rate being shifted to accommodate the different total cannula gas flow rates. This plot demonstrates that a universal correlation can be produced across a range of gas flow rates.

FIGS. 31-35 illustrate portions of a flow regulated nasal delivery system (also referred to as “flow regulated system” or “delivery system” or “system”), according to another embodiment. The system 600 can be used to deliver an oxygen-containing gas to a patient and provide real-time information related to the patient's inspiratory flow rate during the delivery of the gas and can include the same or similar features and function the same or similar as the systems 100, 300 and 400 described above. Thus, some features are not shown and described with reference to this embodiment. As with the previous embodiment, the system 600 can make automatic adjustments to the flow rate of the oxygen-containing gas and/or to the amount or fraction of oxygen within the oxygen-containing gas in real-time during the delivery of the gas.

The system 600 includes a nasal cannula 620, a pressure sensor (not shown, but which can be similar to any of the pressure sensors described herein) and a controller (not shown, but which can be similar to any of the controllers described herein, such as the controller 105). The nasal cannula 620 includes a first nasal prong 622, a second nasal prong 623 and a base portion 636. In this embodiment, the first nasal prong 622 and the second nasal prong 623 are curved shape. The first nasal prong 622 includes a first end portion 650 that defines an inlet opening 626 and a second end portion 652 that defines an outlet opening 628, and a middle portion 654 between the first end portion 650 and the second end portion 652 (see, e.g., FIGS. 31-34). A flow passage 638 is defined by an inner surface of a side wall 624 of the first nasal prong 622 between the inlet opening 626 and the outlet opening 628. The second nasal prong 623 includes a first end portion 651 that defines an inlet opening 627 and a second end portion 653 that defines an outlet opening 629, and a middle portion 655 between the first end portion 651 and the second end portion 653 (see, e.g., FIGS. 31-34). A flow passage 639 is defined by an inner surface of a side wall 625 of the second nasal prong 623 between the inlet opening 627 and the outlet opening 629.

In this embodiment, the side wall 624 of the first nasal prong 622 and the side wall 625 of the second nasal prong 623 can each have constant thickness and a constant inner diameter of the flow passage 638 and the flow passage 639. In alternative embodiments, the side walls 624 and 625 can have a varying thickness and the flow passages 638 and 639 can have a varying diameter in the same or similar manner as described for example, for cannula 320. As described above, FIG. 14C illustrates example inner diameters and wall thicknesses of a nasal prong and example locations for the diameters and thicknesses that can apply to cannula 620.

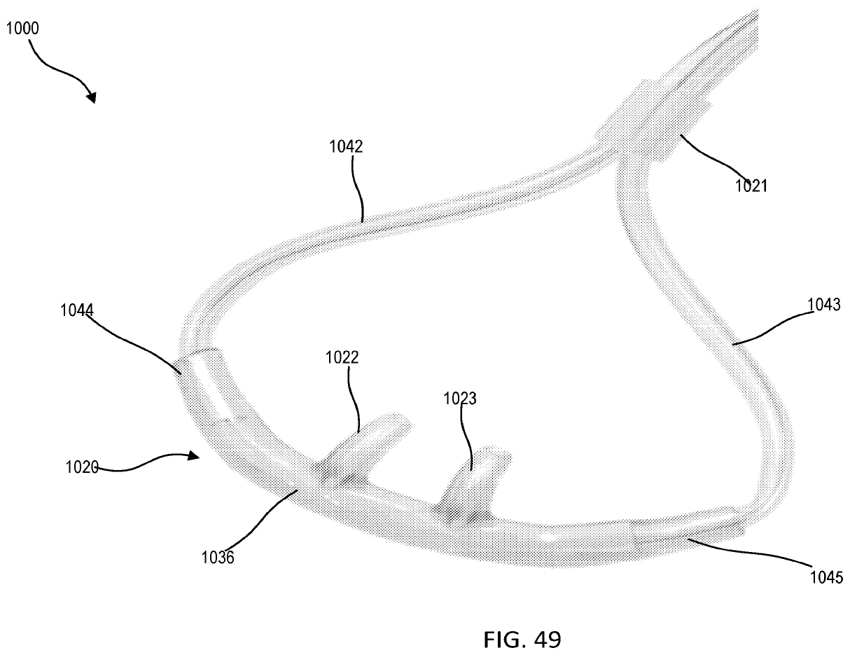

The first nasal prong 622 and the second nasal prong 623 are each configured to be inserted within a nostril of a patient such that the outlet opening 628 and the outlet opening 629 are each disposed within a different nostril of the nose of a user, in the same manner as cannula 320 as shown for example, in FIG. 7. Although not shown, the nasal cannula 620 is couplable to a first support tube and a second support tube as shown for nasal cannula 320 and support tubes 342 and 343 above and as shown in FIG. 49 and described below for nasal cannula 1020. More specifically, a first support tube (not shown) can be coupled to a coupling portion 644 of the base portion 636 and a second support tube (not shown) can be coupled to a coupling portion 645 of the base portion 636. The support tubes can be used to support the nasal cannula 620 on the head of the patient by extending around the ears of the patient. The first support tube and the second support tube can also be coupled together at an adjustment member (e.g., 321, 1021) that can be used to tighten or loosen the support tubes on the patient.

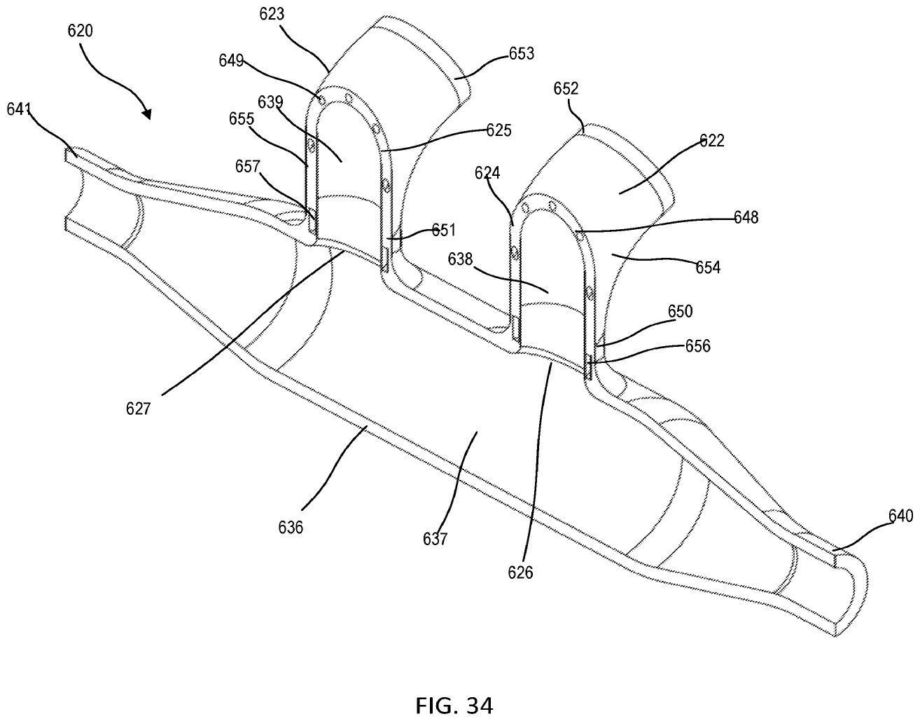

At least one of the support tubes can be fluidically coupled to the source of oxygen-containing gas such that the gas can be delivered to the nasal cannula 620 via the first support tube and/or the second support tube. For example, in some embodiments, the flow passage 638 and the flow passage 639 can each be in fluid communication with one of the first support tube or the second support tube via an interior region 637 of the base portion 636 (see, e.g., FIG. 34). The one of the first coupling portion 644 or the second coupling portion 645 can be coupled to a source of oxygen-containing gas and the other of the first coupling portion 644 or the second coupling portion 645 can be closed similar to the nasal cannulas 320 and 420. The oxygen-containing gas can flow through one of first or second support tube, into the interior region 637 of the base portion 636, into the first flow passage 638 and the second flow passage 639, and into the nostril of the user. The closed coupling portion (644 or 645) prevents the oxygen-containing gas from flowing beyond that coupling portion. In other words, the oxygen-containing gas can only flow into one of the first support tube or the second support tube, and the other of the first support tube and the second support tube is only used to support the nasal cannula 620 on the user.

In an alternative embodiment, both the first coupling portion 644 and the second coupling portion 645 can be fluidically coupled to a source of oxygen-containing gas. In other words, neither of the first coupling portion 644 or second coupling portion 645 are closed. Such an embodiment is shown in FIG. 49 and described below. In such an embodiment, the flow of oxygen-containing gas can be supplied to both the first support tube and the second support tube to flow into the interior region 637, into the first flow passage 638 and the second flow passage 639 and into the nostrils of the user.

Each of the nasal prongs 622 and 623 have multiple ports that can be used to measure pressures associated with the flow of oxygen-containing gas through the flow passages 638 and 639. More specifically, in this embodiment, the side wall 624 of the first nasal prong 622 defines multiple ports 630 spaced apart from each other about an inside circumference within the flow passage 638 at or near the second end portion 652 of the first nasal prong 622. Similarly, the side wall 625 of the second nasal prong 623 defines multiple ports 631 spaced apart from each other about an inside circumference within the flow passage 639 at or near the second end portion 653 of the second nasal prong 623. In this embodiment, there are eight ports 630 and eight ports 631 spaced equally apart from one another, but a different number of ports 630 and ports 631 can alternatively be used depending on the configuration of the nasal cannula.

In some embodiments, each of the ports 630 of the first nasal prong 622 are disposed at a distance from an end surface 618 of the first nasal prong 622 not more than two times a diameter of each (or any) of the ports 630. In other embodiments, each of the ports 630 of the first nasal prong 622 are disposed at a distance from an end surface 618 of the first nasal prong 622 not more than four times a diameter of each (or any) of the ports 630. Similarly, in some embodiments, each of the ports 631 of the second nasal prong 623 are disposed at a distance from an end surface 619 of the second nasal prong 623 not more than two times a diameter of the ports 631. In some embodiments, each of the ports 631 of the second nasal prong 623 are disposed at a distance from the end surface 619 of the second nasal prong 623 not more than four times a diameter of each (or any) of the ports 631. The close proximity of the ports to the end surfaces 618 and 619 of the nasal prongs 622 and 623, respectively, provide for pressure measurements closer to the outlet openings 628, 629 of the nasal prongs 622 and 623, which can result in a more accurate determination of the inspiratory flow rate of the patient based on the pressure measurements. The inclusion of multiple ports also provides for redundancy of measurement of the pressure

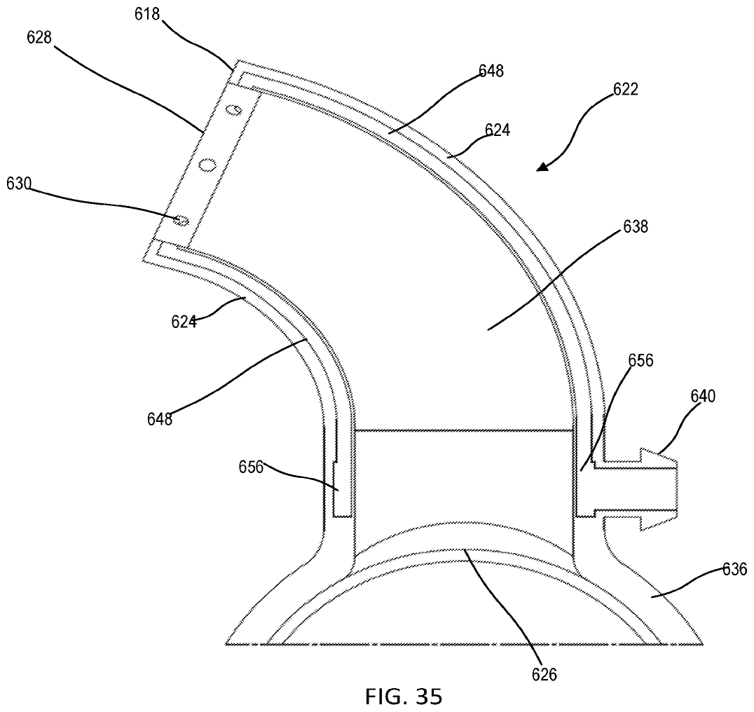

The ports 630 of the first nasal prong 622 can be fluidically coupled to a pressure sensor (not shown in FIGS. 31-35) and the ports 631 of the second nasal prong 623 can be fluidically coupled to a pressure sensor (not shown in FIGS. 31-35) such that pressures associated with the pressures of the oxygen-containing gas within the flow passages 638 and 639 can be measured at the ports 630, 631. For example, each of the ports 630 of the first nasal prong 622 can be in fluid communication with a pressure sensor coupler 640 disposed at the first end portion 650 of the nasal prong 622 via a fluid port channel 648 defined in the side wall 624, as shown in FIG. 35. A circumferential passageway 656 routes the pressurized air from each of the ports 630 to the pressure sensor coupler 640 as shown in FIG. 35. A first pressure sensor tube (not shown) can be coupled to and in fluid communication with the pressure sensor coupler 640 in a similar manner as described above and as shown in FIG. 9.

Similarly, each of the ports 631 of the second nasal prong 623 can be in fluid communication with a pressure sensor coupler 641 disposed at the first end portion 651 of the nasal prong 623 via a fluid port channel (no shown) and circumferential passageway (not shown) to the pressure sensor coupler 641 in the same manner as for nasal prong 622. A second pressure sensor tube (not shown) can be coupled to and in fluid communication with the pressure sensor coupler 641, as described above and shown in FIG. 9. Although not shown, in some embodiments, the pressure sensor tubes can be gathered and coupled together for ease of use on the patient.

The pressure sensor tubes can each be coupled to a pressure sensor (not shown) and used to measure pressures associated with the flow of oxygen-containing gas within the flow passage 638 of the nasal prong 622 and the flow passage 639 of the nasal prong 623. As described above for previous embodiments, pressure measurements can be measured during delivery of the oxygen-containing gas to the nasal cannula 620 and used to determine an inspiratory flow rate of the patient. The ports 630 and 631 at the end surface of the prongs 622, 623 being in close proximity to the outlets 628 and 629 provides for an accurate measure of the pressure of the oxygen-containing gas exiting the prongs 622, 623 and entering the nostrils of the user. The multiple ports 630, 631 provides for redundancy in the pressure measurements and also provides a spatial average of the pressures associated with the flow of the oxygen-containing gas flowing through the prongs 622 and 623 and entering the nostrils. For example, the pressurized air from the multiple ports 630 is aggregated at the pressure sensor connector 640 by circumferential passageway 656 to provide a pressure measurement that is associated with the overall pressure at the outlet 628 based on the pressures at each of the ports 630.

As described above for previous embodiments, a controller (not shown, but which can be similar to any of the controllers described herein) can have a processor (not shown) operatively coupled to the pressure sensors. The processor is configured to produce one or more pressure waveforms characterizing the pressure measurements at each location as a function of time during the delivery of the oxygen-containing gas to the patient in the same manner as described above. Based on at least any of the pressure measurements (or the pressure waveforms produced therefrom), the processor can determine any of an inspiratory flow rate of the patient, a tidal volume of the patient, or a minute ventilation of the patient during the delivery of the oxygen-containing gas. The processor can produce an output based at least in part on the determined inspiratory flow rate, tidal volume, or minute ventilation. For example, the output produced by the processor can include a control signal that causes an adjustment to at least one of a percentage of oxygen within the oxygen-containing gas or a flow rate of the oxygen-containing gas being delivered into the nasal cannula 620 based at least in part on the determined inspiratory flow rate. In some embodiments, the control signal can be sent to a flow control valve (not shown) that is coupled to the source of the oxygen-containing gas and coupled to the nasal cannula 620 to deliver the oxygen-containing gas to the nasal cavity of the patient. With information related to the patient's inspiratory flow rate determined by the processor, adjustments can automatically be made to the flow of the oxygen-containing gas in real-time during the delivery of the oxygen-containing gas. As also described above for previous embodiments, the processor can receive input including data related to a health parameter associated with the patient. In such a case, the processor can produce the control signal based at least in part on the determined inspiratory flow rate and the health parameter(s).

FIGS. 36-40 illustrate portions of a flow regulated nasal delivery system (also referred to as “flow regulated system” or “delivery system” or “system”), according to another embodiment. The system 700 can be used to deliver an oxygen-containing gas to a patient and provide real-time information related to the patient's inspiratory flow rate during the delivery of the gas and can include the same or similar features and function the same or similar as the systems 100, 300, 400 and 600 described above. Thus, some features are not shown and described with reference to this embodiment. As with the previous embodiment, the system 700 can make automatic adjustments to the flow rate of the oxygen-containing gas and/or to the amount or fraction of oxygen within the oxygen-containing gas in real-time during the delivery of the gas.

The system 700 includes a nasal cannula 720, a pressure sensor (not shown, but which can be similar to any of the pressure sensors described herein) and a controller (not shown, but which can be similar to any of the controllers described herein, such as the controller 105). The nasal cannula 720 includes a first nasal prong 722, a second nasal prong 723 and a base portion 736. In this embodiment, the first nasal prong 722 and the second nasal prong 723 are curved shape. The first nasal prong 722 includes a first end portion 750 that defines an inlet opening 726 and a second end portion 752 that defines an outlet opening 728, and a middle portion 754 between the first end portion 750 and the second end portion 752 (see, e.g., FIGS. 36, 37 and 39). A flow passage 738 is defined by an inner surface of a side wall 724 of the first nasal prong 722 between the inlet opening 726 and the outlet opening 728. The second nasal prong 723 includes a first end portion 751 that defines an inlet opening 727 and a second end portion 753 that defines an outlet opening 729, and a middle portion 755 between the first end portion 751 and the second end portion 753 (see, e.g., FIGS. 36, 37 and 39). A flow passage 739 is defined by an inner surface of a side wall 725 of the second nasal prong 723 between the inlet opening 727 and the outlet opening 729.

In this embodiment, the side wall 724 of the first nasal prong 722 and the side wall 725 of the second nasal prong 723 can each have constant thickness and a constant inner diameter of the flow passage 738 and the flow passage 739. In alternative embodiments, the side walls 724 and 725 can have a varying thickness and the flow passages 738 and 739 can have a varying diameter in the same or similar manner as described for example, for cannula 320. As described above, FIG. 14C illustrates example inner diameters and wall thicknesses of a nasal prong and example locations for the diameters and thicknesses that can apply to cannula 720.

The first nasal prong 722 and the second nasal prong 723 are each configured to be inserted within a nostril of a patient such that the outlet opening 728 and the outlet opening 729 are each disposed within a different nostril of the nose of a user, in the same manner as cannula 320 as shown for example, in FIG. 7. Although not shown, the nasal cannula 720 is couplable to a first support tube and a second support tube as shown for nasal cannula 320 and support tubes 342 and 343 above and as shown in FIG. 49 and described below for nasal cannula 1020. More specifically, a first support tube (not shown) can be coupled to a coupling portion 744 of the base portion 736 and a second support tube (not shown) can be coupled to a coupling portion 745 of the base portion 736. The support tubes can be used to support the nasal cannula 720 on the head of the patient by extending around the ears of the patient. The first support tube and the second support tube can also be coupled together at an adjustment member (e.g., 321, 1021) that can be used to tighten or loosen the support tubes on the patient.

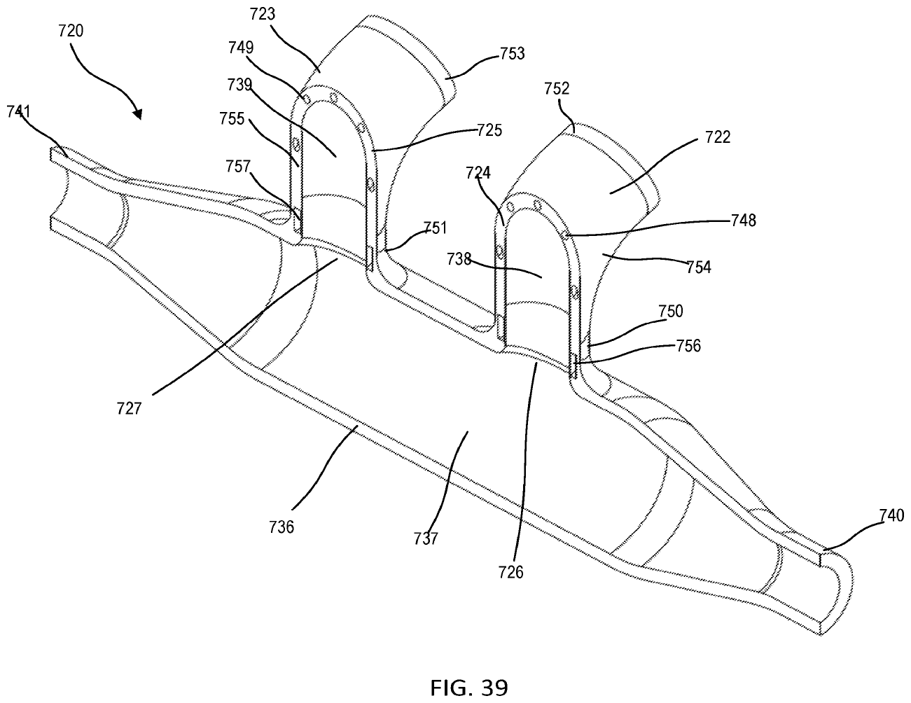

At least one of the support tubes can be fluidically coupled to the source of oxygen-containing gas such that the gas can be delivered to the nasal cannula 720 via the first support tube and/or the second support tube. For example, in some embodiments, the flow passage 738 and the flow passage 739 can each be in fluid communication with one of the first support tube or the second support tube via an interior region 737 of the base portion 736 (see, e.g., FIG. 39). The one of the first coupling portion 744 or the second coupling portion 745 can be coupled to a source of oxygen-containing gas and the other of the first coupling portion 744 or the second coupling portion 745 can be closed. The oxygen-containing gas can flow through one of first or second support tube, into the interior region 737 of the base portion 736, into the first flow passage 738 and the second flow passage 739, and into the nostril of the user. The closed coupling portion (744 or 745) prevents the oxygen-containing gas from flowing beyond that coupling portion. In other words, in this embodiment, the oxygen-containing gas only flows into one of the first support tube or the second support tube, and the other of the first support tube and the second support tube is only used to support the nasal cannula 720 on the user.

In an alternative embodiment, both the first coupling portion 744 and the second coupling portion 745 can be fluidically coupled to a source of oxygen-containing gas. In other words, neither of the first coupling portion 744 or second coupling portion 745 are closed. Such an embodiment is shown in FIG. 49 and described below. In such an embodiment, the flow of oxygen-containing gas can be supplied to both the first support tube and the second support tube to flow into the interior region 737, into the first flow passage 738 and the second flow passage 739 and into the nostrils of the user.

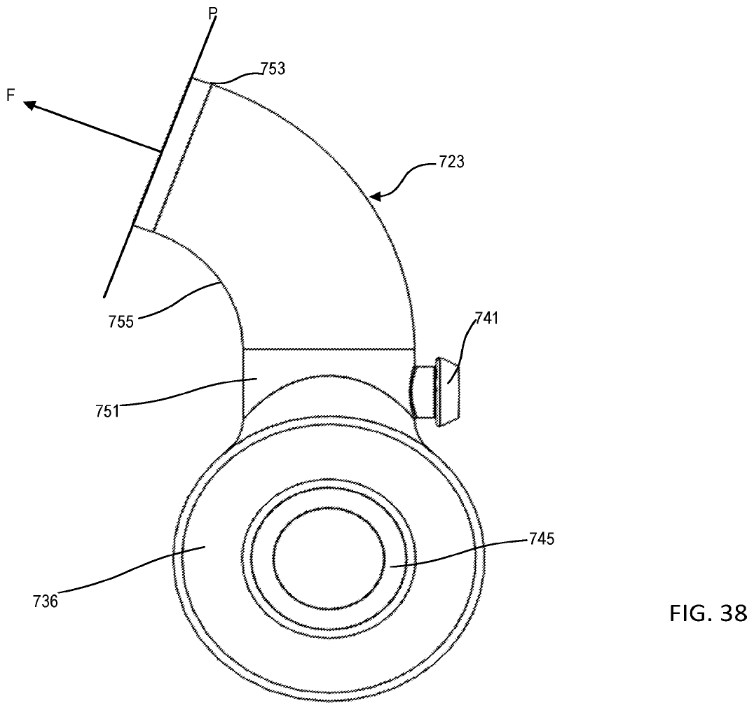

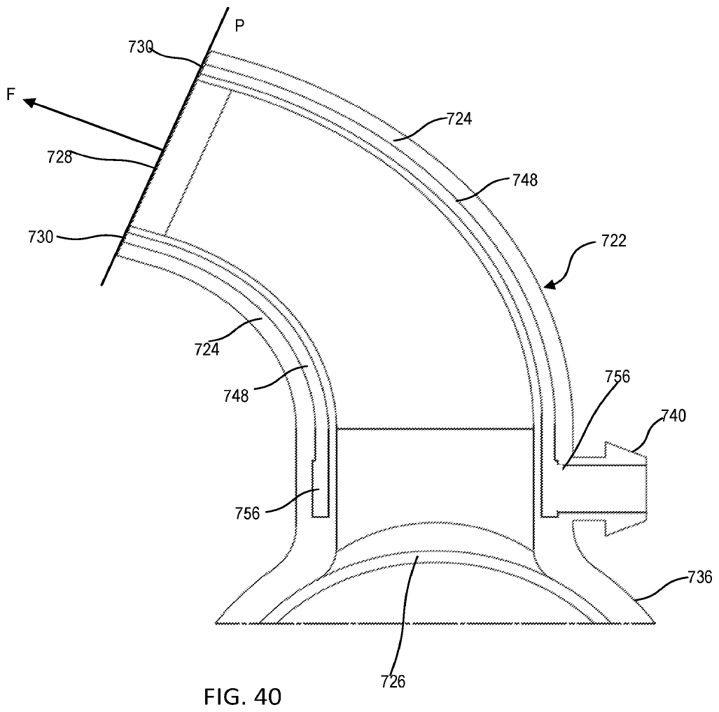

Each of the nasal prongs 722 and 723 have multiple ports that can be used to measure pressures associated with the flow of oxygen-containing gas through the flow passages 738 and 739. In this embodiment, the multiple ports for each of the nasal prongs 722, 723 are defined at the second end portions 752 and 753 of the respective nasal prong 722 and 723. More specifically, in this embodiment, the side wall 724 of the first nasal prong 722 defines multiple ports 730 at an end surface 718 (see, e.g., FIGS. 36, 37, and 40). Similarly, the side wall 725 of the first nasal prong 723 defines multiple ports 731 at an end surface 719 (see, e.g., FIGS. 36, 37, and 40). In this embodiment, there are eight ports 730 and eight ports 731, but a different number of ports 730 and ports 731 can alternatively be used depending on the particular configuration of the nasal cannula. As shown in FIG. 36, the ports 730 and the ports 731 are spaced evenly about circumference of the end surfaces 718 and 719 respectively. The location of the ports 730, 731 on the end surfaces 718 and 719 of the nasal prongs 722 and 723, respectively, provide for pressure measurements closer to the outlet openings 728 and 729 of the nasal prongs 722 and 723, and in direct alignment with the exhalation air from the nose, which can result in a more accurate determination of the inspiratory flow rate of the patient based on the pressure measurements. The inclusion of multiple ports 730, 731 about the circumference of the end surfaces 718, 719 provides for redundancy of the measurement of the pressures at the ports 730. 731. As shown in FIGS. 38 and 40, in this embodiment, the end surfaces 718, 719 are disposed in a plane P that is perpendicular to the flow F of the oxygen-containing gas and therefore, the ports 730, 731 are also disposed perpendicular to the flow F. This positioning of the ports 730. 731 can provide for the exhaled air from the nose to be substantially aligned with the location of the ports 730, 731, and increase an accuracy of the pressure measurement associated with the pressures at the ports 730, 731. Specifically, by having the exhaled air directly impact the ports (i.e., the exhaled airflow is substantially aligned with a longitudinal centerline of the port), the measurement of the pressure (including a stagnation pressure produced by the exhaled air) may be more accurately measured.

The ports 730 of the first nasal prong 722 can be fluidically coupled to a pressure sensor (not shown in FIGS. 36-40) and the ports 731 of the second nasal prong 723 can be fluidically coupled to a pressure sensor (not shown in FIGS. 36-40) such that pressures associated with the pressures of the oxygen-containing gas within the flow passages 738 and 739 can be measured at the ports 730, 731. For example, each of the ports 730 of the first nasal prong 722 can be in fluid communication with a pressure sensor coupler 740 disposed at the first end portion 750 of the nasal prong 722 via a fluid port channel 748 defined in the side wall 724, as shown in FIG. 39. A circumferential passageway 656 routes the flow of pressurized air from each of the ports 730 to the pressure sensor coupler 740 as shown in FIG. 39. A first pressure sensor tube (not shown) can be coupled to and in fluid communication with the pressure sensor coupler 740 in a similar manner as described above and as shown in FIG. 9.

Similarly, each of the ports 731 of the second nasal prong 723 can be in fluid communication with a separate pressure sensor coupler 741 disposed at the first end portion 751 of the nasal prong 723 via a fluid port channel (no shown) and circumferential passageway (not shown) to the pressure sensor coupler 741 in the same manner as for nasal prong 722. A second pressure sensor tube (not shown) can be coupled to and in fluid communication with the pressure sensor coupler 741, as described above and shown in FIG. 9. Although not shown, in some embodiments, the pressure sensor tubes can be gathered and coupled together for ease of use on the patient.

The pressure sensor tubes can each be coupled to a pressure sensor (not shown) and used to measure pressures associated with the flow of oxygen-containing gas within the passage 738 of the nasal prong 722 and the flow passage 739 of the nasal prong 723. As described above for previous embodiments, pressure measurements can be measured during delivery of the oxygen-containing gas to the nasal cannula 720 and used to determine an inspiratory flow rate of the patient. The ports 730 and 731 at the end surface of the prongs 722, 723 being in close proximity to the outlets 728 and 729 provides for a more accurate measure of the pressure of the oxygen-containing gas exiting the prongs 722, 723 and entering the nostrils of the user. The multiple ports 730, 731 provides for redundancy in the pressure measurements and also provides a spatial average of the pressures associated with the flow of the oxygen-containing gas flowing through the prongs 722 and 723 and entering the nostrils. For example, the flow of pressurized air through the multiple ports 730 is aggregated at the pressure sensor connector 740 by the circumferential passageway 756 to provide a pressure measurement that is associated with the overall pressures at the outlet 728 based on the pressures at each of the ports 730.

As described above for previous embodiments, a controller (not shown, but which can be similar to any of the controllers described herein) can have a processor (not shown) operatively coupled to the pressure sensors. The processor is configured to produce one or more pressure waveforms characterizing the pressure measurements at each location as a function of time during the delivery of the oxygen-containing gas to the patient in the same manner as described above. Based on at least any of the pressure measurements (or the pressure waveforms produced therefrom), the processor can determine any of an inspiratory flow rate of the patient, a tidal volume of the patient, or a minute ventilation of the patient during the delivery of the oxygen-containing gas. The processor can produce an output based at least in part on the determined inspiratory flow rate, tidal volume, or minute ventilation. For example, the output produced by the processor can include a control signal that causes an adjustment to at least one of a percentage of oxygen within the oxygen-containing gas or a flow rate of the oxygen-containing gas being delivered into the nasal cannula 720 based at least in part on the determined inspiratory flow rate. In some embodiments, the control signal can be sent to a flow control valve (not shown) that is coupled to the source of the oxygen-containing gas and coupled to the nasal cannula 720 to deliver the oxygen-containing gas to the nasal cavity of the patient. With information related to the patient's inspiratory flow rate determined by the processor, adjustments can automatically be made to the flow of the oxygen-containing gas in real-time during the delivery of the oxygen-containing gas. As also described above for previous embodiments, the processor can receive input including data related to a health parameter associated with the patient. In such a case, the processor can produce the control signal based at least in part on the determined inspiratory flow rate and the health parameter(s).

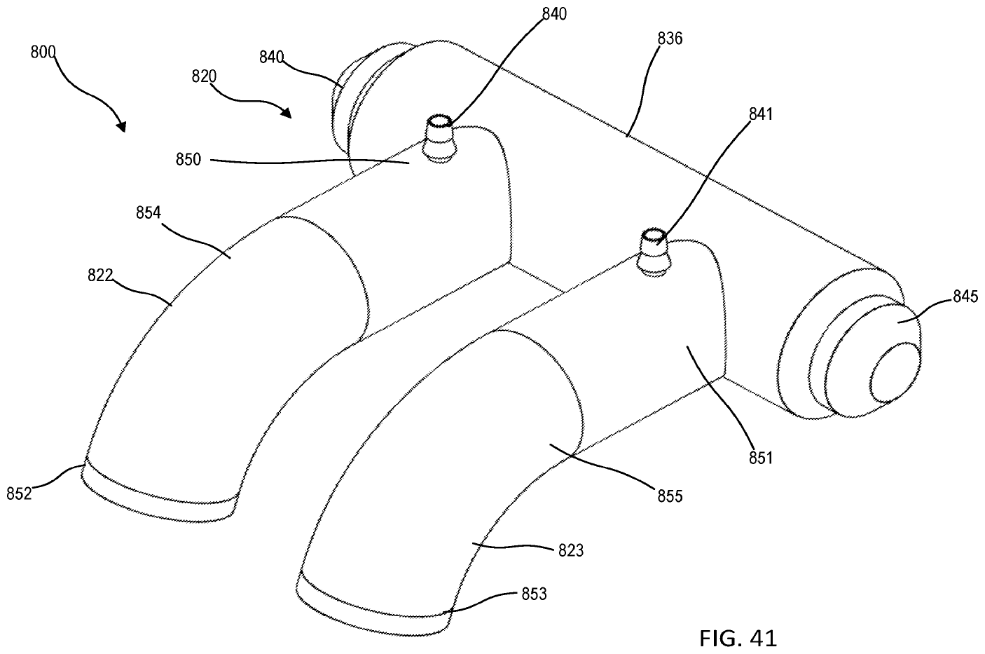

FIGS. 41-46 illustrate portions of a flow regulated nasal delivery system (also referred to as “flow regulated system” or “delivery system” or “system”), according to another embodiment. The system 800 can be used to deliver an oxygen-containing gas to a patient and provide real-time information related to the patient's inspiratory flow rate during the delivery of the gas and can include the same or similar features and function the same or similar as the systems 100, 300, 400, 600 and 700 described above. Thus, some features are not shown and described with reference to this embodiment. As with the previous embodiment, the system 800 can make automatic adjustments to the flow rate of the oxygen-containing gas and/or to the amount or fraction of oxygen within the oxygen-containing gas in real-time during the delivery of the gas.

The system 800 includes a nasal cannula 820, a pressure sensor (not shown) and a controller (not shown). The nasal cannula 820 includes a first nasal prong 822, a second nasal prong 823 and a base portion 836. In this embodiment, the first nasal prong 822 and the second nasal prong 823 are curved shape. The first nasal prong 822 includes a first end portion 850 that defines an inlet opening 826 and a second end portion 852 that defines an outlet opening 828. and a middle portion 854 between the first end portion 850 and the second end portion 852 (see, e.g., FIGS. 41, 42 and 44). A flow passage 838 is defined by an inner surface of a side wall 824 of the first nasal prong 822 between the inlet opening 826 and the outlet opening 828. The second nasal prong 823 includes a first end portion 851 that defines an inlet opening 827 and a second end portion 853 that defines an outlet opening 829, and a middle portion 855 between the first end portion 851 and the second end portion 853 (see, e.g., FIGS. 1, 42 and 44). A flow passage 839 is defined by an inner surface of a side wall 825 of the second nasal prong 823 between the inlet opening 827 and the outlet opening 829.

In this embodiment, the side wall 824 of the first nasal prong 822 and the side wall 825 of the second nasal prong 823 can each have constant thickness and a constant inner diameter of the flow passage 838 and the flow passage 839. In alternative embodiments, the side walls 824 and 825 can have a varying thickness and the flow passages 838 and 839 can have a varying diameter in the same or similar manner as described for example, for cannula 320. As described above, FIG. 14C illustrates example inner diameters and wall thicknesses of a nasal prong and example locations for the diameters and thicknesses that can apply to cannula 820.

The first nasal prong 822 and the second nasal prong 823 are each configured to be inserted within a nostril of a patient such that the outlet opening 828 and the outlet opening 829 are each disposed within a different nostril of the nose of a user, in the same manner as cannula 320 as shown for example, in FIG. 7. Although not shown, the nasal cannula 820 is couplable to a first support tube and a second support tube as shown for nasal cannula 320 and support tubes 342 and 343 above and as shown in FIG. 49 and described below for nasal cannula 1020. More specifically, a first support tube (not shown) can be coupled to a coupling portion 844 of the base portion 836 and a second support tube (not shown) can be coupled to a coupling portion 845 of the base portion 836. The support tubes can be used to support the nasal cannula 820 on the head of the patient by extending around the ears of the patient. The first support tube and the second support tube can also be coupled together at an adjustment member that can be used to tighten or loosen the support tubes on the patient.

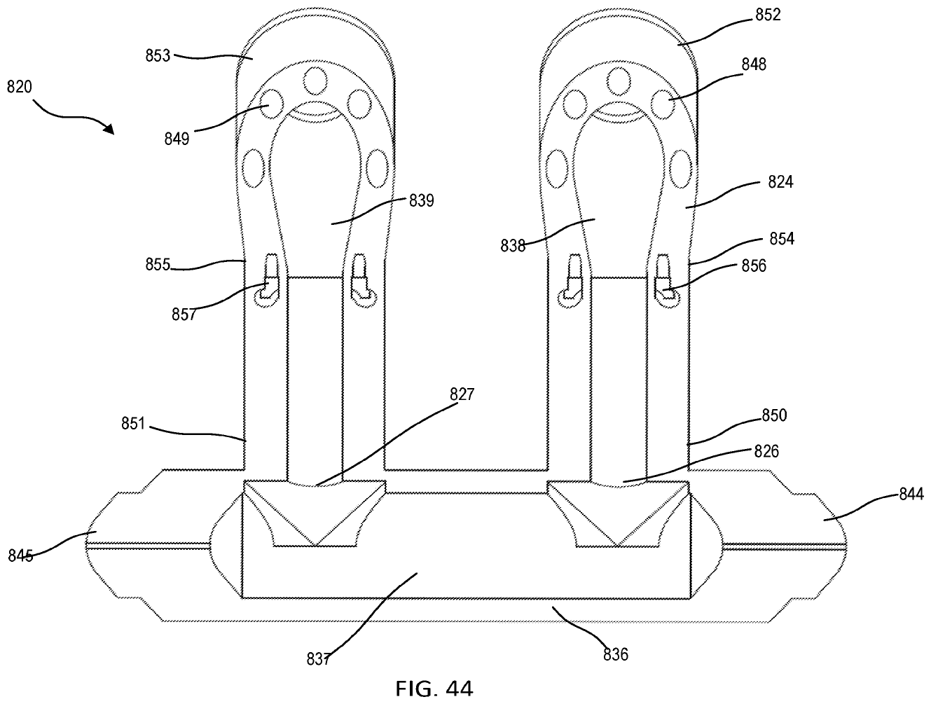

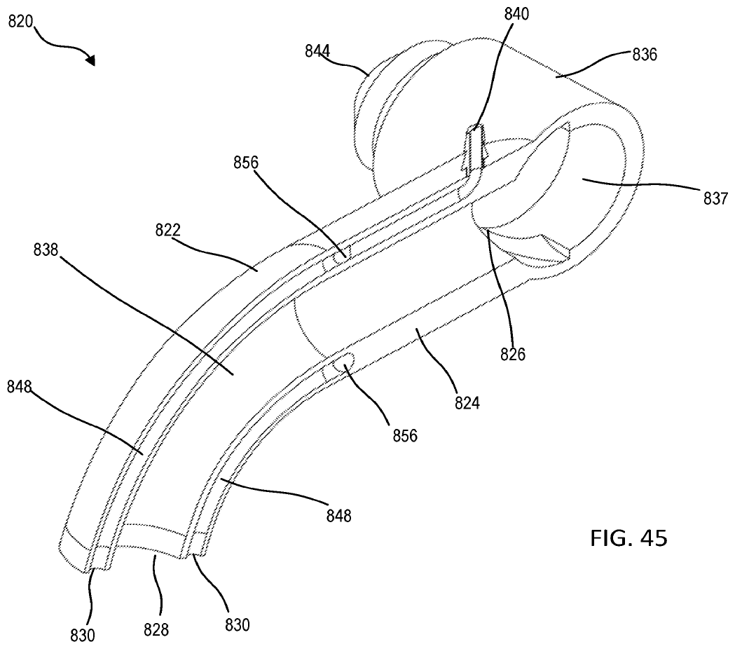

At least one of the support tubes can be fluidically coupled to the source of oxygen-containing gas such that the gas can be delivered to the nasal cannula 820 via the first support tube and/or the second support tube. For example, in some embodiments, the flow passage 838 and the flow passage 839 can each be in fluid communication with one of the first support tube or the second support tube via an interior region 837 of the base portion 836 (see, e.g., FIGS. 44 and 45). The one of the first coupling portion 844 or the second coupling portion 845 can be coupled to a source of oxygen-containing gas and the other of the first coupling portion 844 or the second coupling portion 845 can be closed. The oxygen-containing gas can flow through one of first or second support tube, into the interior region 837 of the base portion 836, into the first flow passage 738 and the second flow passage 839, and into the nostril of the user. The closed coupling portion (844 or 845) prevents the oxygen-containing gas from flowing beyond that coupling portion. In other words, the oxygen-containing gas can only flow into one of the first support tube or the second support tube. and the other of the first support tube and the second support tube is only used to support the nasal cannula 820 on the user.

In an alternative embodiment, both the first coupling portion 844 and the second coupling portion 845 can be fluidically coupled to a source of oxygen-containing gas. In other words, neither of the first coupling portion 844 or the second coupling portion 845 are closed. Such an embodiment is shown in FIG. 49 and described below. In such an embodiment, the flow of oxygen-containing gas can be supplied to both the first support tube and the second support tube to flow into the interior region 837, into the first flow passage 838 and the second flow passage 839 and into the nostrils of the user.

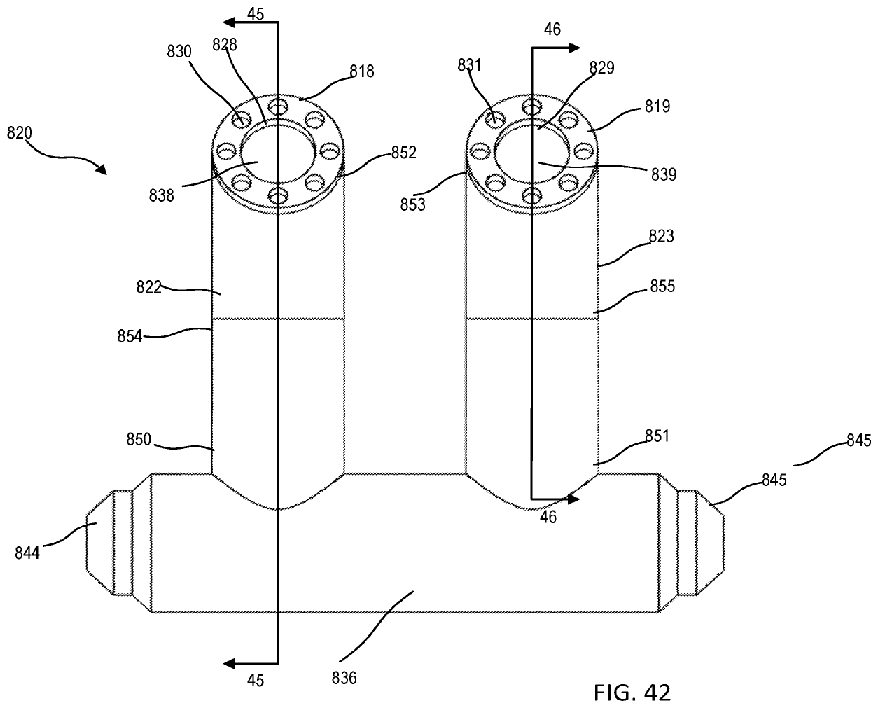

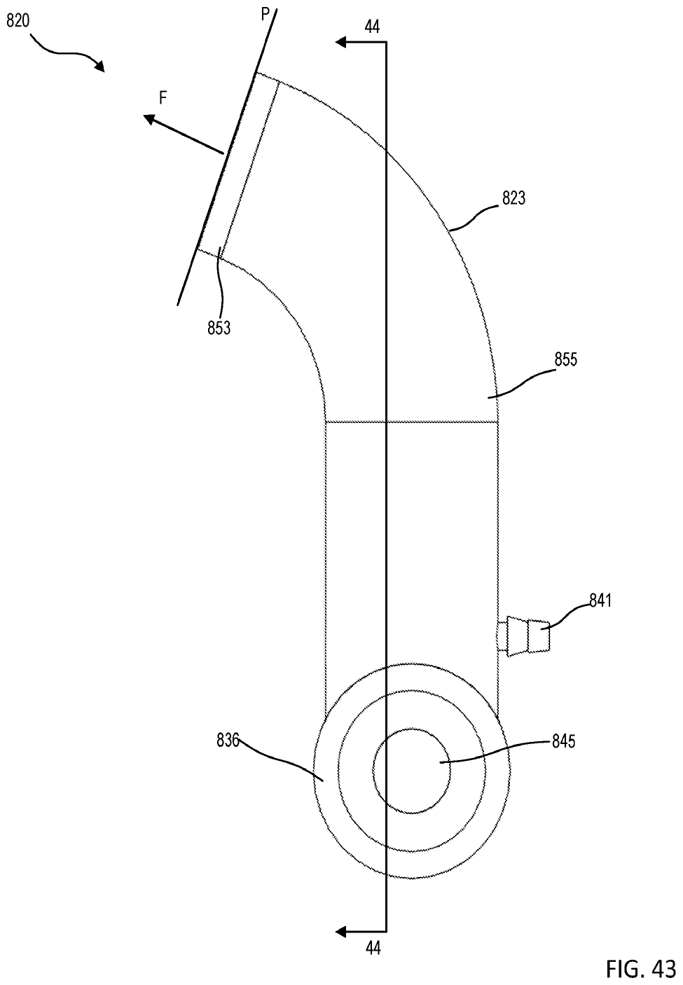

Each of the nasal prongs 822 and 823 have multiple ports that can be used to measure pressures associated with the flow of oxygen-containing gas through the flow passages 838 and 839. In this embodiment, the multiple ports for each of the nasal prongs 822, 823 are defined at the second end portions 852 and 853 of the respective nasal prong 822 and 823. More specifically, in this embodiment, the side wall 824 of the first nasal prong 822 defines multiple ports 830 at an end surface 818 (see, e.g., FIG. 42). Similarly, the side wall 825 of the first nasal prong 823 defines multiple ports 831 at an end surface 819 (see, e.g., FIG. 42). In this embodiment, there are eight ports 830 and eight ports 831, but a different number of ports 830 and ports 831 can alternatively be used depending on the configuration of the nasal cannula. As shown in FIG. 42, the ports 830 and the ports 831 are spaced evenly about circumference of the end surfaces 818 and 819 respectively. The location of the ports 830, 831 on the end surfaces 818 and 819 of the nasal prongs 822 and 823, respectively, provide for pressure measurements closer to the outlet openings 828 and 829 of the nasal prongs 822 and 823, which can result in a more accurate determination of the inspiratory flow rate of the patient based on the pressure measurements. The inclusion of multiple ports 830, 831 about the circumference of the end surfaces 818, 819 provides for redundancy of the measurement of the pressures at the ports 830, 831. As shown in FIG. 43, in this embodiment, the end surfaces 818, 819 are disposed in a plane P that is perpendicular to the flow F of the oxygen-containing gas and therefore, the ports 830, 831 are also disposed perpendicular to the flow F. This positioning of the ports 830, 831 can provide for the exhaled air from the nose to be substantially aligned with the location of the ports 830, 831, which can increase an accuracy of the pressure measurement associated with the pressures at the ports 830, 831. Specifically, by having the exhaled air directly impact the ports (i.e., the exhaled airflow is substantially aligned with a longitudinal centerline of the port), the measurement of the pressure (including a stagnation pressure produced by the exhaled air) may be more accurately measured.

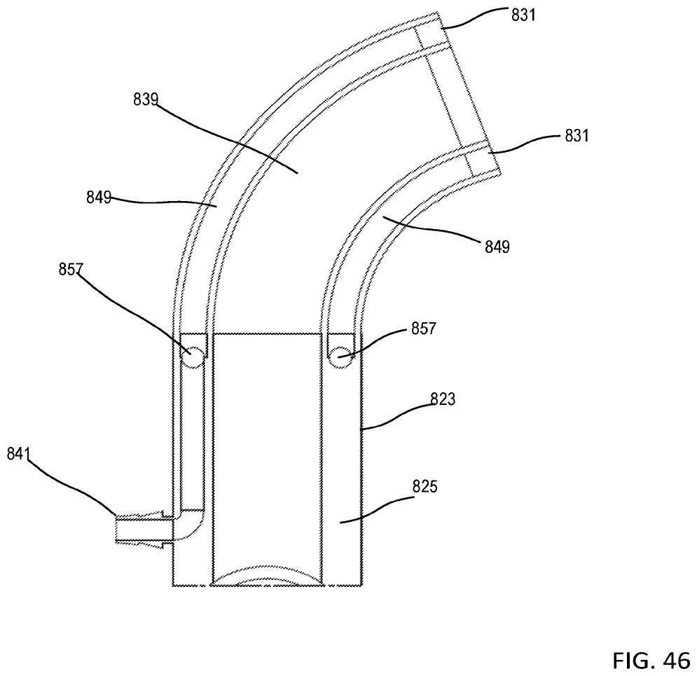

The ports 830 of the first nasal prong 822 can be fluidically coupled to a pressure sensor (not shown in FIGS. 41-46) and the ports 831 of the second nasal prong 823 can be fluidically coupled to a pressure sensor (not shown in FIGS. 41-46) such that pressures associated with the pressures of the oxygen-containing gas within the flow passages 838 and 839 can be measured at the ports 830, 831. For example, each of the ports 830 of the first nasal prong 822 can be in fluid communication with a pressure sensor coupler 840 disposed at the first end portion 850 of the nasal prong 822 via a fluid port channel 848 defined in the side wall 824, as shown in FIGS. 45 and 46. A circumferential passageway 856 routes the flow of pressurized air from each of the ports 830 to the pressure sensor coupler 840 as shown in FIGS. 45 and 46.

A separate pressure sensor tube (not shown) can be coupled to and in fluid communication with the pressure sensor coupler 840 in a similar manner as described above and as shown in FIG. 9. Similarly, each of the ports 831 of the second nasal prong 823 can be in fluid communication with a separate pressure sensor coupler 841 disposed at the first end portion 851 of the nasal prong 823 via a fluid port channel (no shown) and circumferential passageway (not shown) to the pressure sensor coupler 841 in the same manner as for nasal prong 822. A separate pressure sensor tube (not shown) can be coupled to and in fluid communication with the pressure sensor coupler 841, as described above and shown in FIG. 9. Although not shown, in some embodiments, the pressure sensor tubes can be gathered and coupled together for ease of use on the patient.

The pressure sensor tubes can each be coupled to a pressure sensor (not shown) and used to measure pressures associated with the flow of oxygen-containing gas within the passage 838 of the nasal prong 822 and the flow passage 839 of the nasal prong 823. As described above for previous embodiments, pressure measurements can be measured during delivery of the oxygen-containing gas to the nasal cannula 820 and used to determine an inspiratory flow rate of the patient. The ports 830 and 831 at the end surface of the prongs 822, 823 being in close proximity to the outlets 828 and 829 provides for an accurate measure of the pressure of the oxygen-containing gas exiting the prongs 822, 823 and entering the nostrils of the user. The multiple ports 830, 831 provides for redundancy in the pressure measurements and also provides a spatial average of the pressures associated with the flow of the oxygen-containing gas flowing through the prongs 822 and 823 and entering the nostrils. For example, the flow of pressurized air through the multiple ports 830 is aggregated at the pressure sensor connector 840 by the circumferential passageway 856 to provide a pressure measurement that is associated with the overall pressure at the outlet 828 based on the pressures at each of the ports 830.

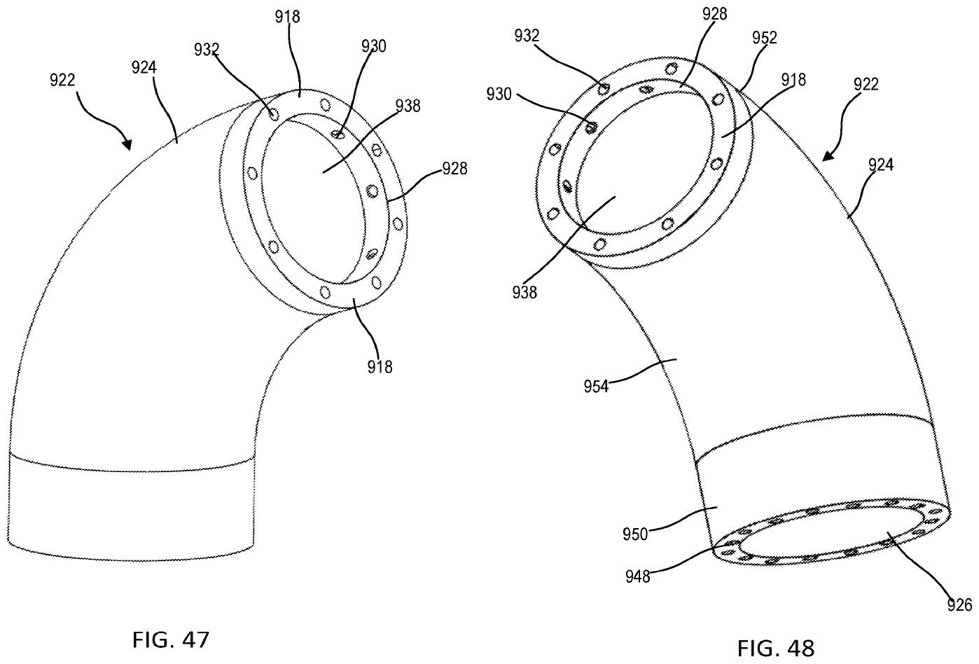

FIGS. 47 and 48 illustrate an alternative prong that can be included within a flow regulated nasal delivery system (also referred to as “flow regulated system” or “delivery system” or “system”), described herein. A nasal prong 922 can be constructed similar to or the same as and can function the same or similar to other embodiments of a nasal prong described herein. Thus, some features of the nasal prong 922 and its functions are not shown and described with reference to this embodiment.

The nasal prong 922 is curved shape and has a first end portion 950, a middle portion 954 and a second end portion 952. The nasal prong 922 has a side wall 924 that defines an inlet opening 926, an outlet opening 927 and a flow passage 938 is defined by an inner surface of the side wall 924 between the inlet opening 926 and the outlet opening 927.

In this embodiment, the side wall 924 has a constant thickness and a constant inner diameter of the flow passage 938. In alternative embodiments, the side wall 924 can have a varying thickness and the flow passage 938 can have a varying diameter in the same or similar manner as described for example, for cannula 320. As described above, FIG. 14C illustrates example inner diameters and wall thicknesses of a nasal prong and example locations for the diameters and thicknesses that can apply to the nasal prong 922.

The nasal prong 922 can be coupled to or formed integrally with a base portion (not shown) as described for previous embodiments. The nasal prong 922 is configured to be inserted within a nostril of a patient such that the outlet opening 928 is disposed in a nostril of the nose of a user, in the same manner as cannula 320 as shown for example, in FIG. 7.

In this embodiment, the nasal prong 922 includes multiple ports 930 and 932 defined by the side wall 924 at the second end portion 952. The ports 930 and 931 can be used to measure pressures associated with the flow of oxygen-containing gas through the flow passage 938. The ports 930 are spaced apart from each other about an inside circumference within the flow passage 938 at or near the second end portion 952. The ports 931 are defined at an end surface 918 of the nasal prong 922 and are spaced evenly about a circumference of the end surface 918. In this embodiment, there are eight ports 930 and eight ports 931 spaced equally apart from one another, but a different number of ports 930 and 931 can alternatively be used depending on the configuration of the nasal cannula.

As described above for previous embodiments, the ports 930 and 931 can be fluidically coupled to a pressure sensor (not shown in FIGS. 47-48) such that pressures associated with the pressures of the oxygen-containing gas within the flow passages 938 can be measured as the ports 930, 931. For example, each of the ports 930 and 931 can be in fluid communication with a pressure sensor coupler (not shown) disposed at the first end portion 950 of the nasal prong 922 via a fluid port channel 948 defined in the side wall 924. A circumferential passageway (not shown) routes the flow of pressurized air to the pressure sensor coupler.

FIG. 49 illustrates a nasal cannula 1020 of a flow regulated oxygen delivery system shown coupled to support tubes 1042 and 1043. The nasal cannula 1020 can be part of a flow regulated nasal delivery system (also referred to as “flow regulated system” or “delivery system” or “system”), as described herein. The nasal cannula 1020 includes a first nasal prong 1022, a second nasal prong 1023 and a base portion 1036. The nasal cannula 1022 can be configured the same or similar to and function the same as or similar to the nasal cannulas described herein. For example, the nasal prongs 1022 and 1023 can include one or more openings that can be fluidically coupled to a pressure sensor as described herein.

In this embodiment, the base portion 1036 includes coupling portions 1044 and 1045 that can be coupled to the support tubes 1042 and 1043, respectively. The support tubes 1042 and 1043 can be used to support the nasal cannula 1020 on the head of the patient by extending around the ears of the patient. The first support tube 1042 and the second support tube 1043 can also be coupled together at an adjustment member 1021 that can be used to tighten or loosen the support tubes on the patient. The support tubes 1042 and 1043 can each be fluidically coupled to a source of oxygen-containing gas to deliver a flow of oxygen-containing gas to the nasal cannula through each of the coupling portions 1044 and 1045.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

Although various embodiments have been described as having particular features and/or combinations of components, other embodiments are possible having various combinations or subcombinations of any features and/or components from any of the embodiments described herein. For example, any of the embodiments of a nasal cannula can include one, two, three or a different number of ports that can be used to measure pressures within the flow passages of the nasal cannula.

1-13. (Canceled)

14. A system for delivering a flow of oxygen-containing gas to an airway of a patient, comprising:
a nasal cannula having at least one nasal prong, the at least one nasal prong having a first end portion defining an inlet opening and a second end portion defining an outlet opening and having a side wall defining a flow passage between the first end portion and the second end portion, the at least one nasal prong configured to be inserted within a nostril of a patient such that the outlet opening is disposed within the nostril, the side wall of the at least one nasal prong defining a port;
the nasal cannula configured to be removably couplable to a source of the oxygen-containing gas such that the oxygen-containing gas can be delivered to the nasal cannula and into the airway of the patient via the nasal prong;
a pressure sensor operably couplable to the nasal cannula, the pressure sensor configured to measure a pressure associated with the flow passage during a time period; and
a controller having a processor operatively coupled to the pressure sensor and configured to:
produce a pressure waveform characterizing the pressure as a function of time during the delivery of the oxygen-containing gas;
determine, based on the pressure waveform an inspiratory flow rate of the patient;
determine, based on the inspiratory flow rate, at least one of a tidal volume or a minute ventilation; and
producing an output based at least in part on at least one of the determined inspiratory flow rate, the tidal volume, or the minute ventilation.

15. The system of claim 14, wherein:
the output includes a control signal that causes an adjustment to at least one of a percentage of oxygen within the oxygen-containing gas or a flow rate of the oxygen-containing gas being delivered into the nasal cannula.

16. The system of claim 15, further comprising:
a flow control valve operatively coupled to the processor and to the source of the oxygen-containing gas, the flow control valve configured to receive the control signal to control at least one of the percentage of oxygen within the oxygen-containing gas or the flow rate of the oxygen-containing gas delivered into the nasal cannula.

17. The system of claim 16, wherein:
the processor is configured to receive input including data related to a health parameter associated with the patient;
the processor configured to produce the control signal based at least in part on the health parameter; and
the control signal is received by the flow control valve such that the adjustment to at least one of the percentage of oxygen within the oxygen-containing gas or the flow rate of the oxygen-containing gas being delivered into the nasal cannula is based at least in part on the determined inspiratory flow rate and the health parameter.

18. The system of claim 17, wherein the health parameter is an oxygen saturation value.

19. A method for delivering oxygen-containing gas to an airway of a patient, comprising:
providing a first flow of an oxygen-containing gas to a nasal cannula having at least one nasal prong inserted within a nostril of the patient, the nasal prong having a first end portion defining an inlet opening and a second end portion defining an outlet opening and defining a flow passage between the inlet opening and the outlet opening, the first flow of the oxygen-containing gas having a first flow rate and a first percentage of oxygen within the oxygen-containing gas;
measuring a plurality of pressure measurements associated with the first flow of the oxygen-containing gas through the flow passage of the nasal prong via a pressure sensor over a time period;
communicating the plurality of pressure measurements to a controller operatively coupled to the pressure sensor and a source of the oxygen-containing gas, the controller including a processor;
determining, at the processor, a pressure waveform characterizing the plurality of pressure measurements as a function of time during the first flow of the oxygen-containing gas;
based on the pressure waveform, determining at the processor at least one of an inspiratory flow rate of the patient, a tidal volume of the patient, or a minute ventilation of the patient;
producing, at the processor, a control signal based at least on at least one of the inspiratory flow rate, the tidal volume, or the minute ventilation; and
sending the control signal to the source of the oxygen-containing gas to adjust at least one of a percentage of oxygen within the oxygen-containing gas or a flow rate of the oxygen-containing gas being delivered into the nasal cannula via the first flow, thereby producing a second flow of the oxygen-containing gas.

20. The method of claim 19, wherein the control signal causes a flow control valve operably coupled to the processor and coupled to the source of the oxygen-containing gas to adjust at least one of the percentage of oxygen within the oxygen-containing gas or the flow rate of the oxygen-containing gas being delivered into the nasal cannula.

21. The method of claim 20, wherein the processor is configured to receive input from a user including data related to a health parameter associated with the patient, and
the control signal produced by the processor is based further on the health parameter associated with the patient.

22. An apparatus for delivering a flow of oxygen-containing gas to an airway of a patient, comprising:
a nasal cannula having a nasal prong, the nasal prong including a first end portion defining an inlet opening, a second end portion defining outlet opening, and a middle portion between the first end portion and the second end portion, a side wall of the nasal prong defining a flow passage between the inlet opening and the outlet opening, the nasal prong configured to be inserted within a nostril of a patient such that the outlet opening is disposed within the nostril,
the inlet opening of the nasal prong configured to be fluidically coupled to a support tube to deliver a flow of the oxygen-containing gas into the airway of the patient via the nasal prong,
the side wall of the nasal prong defining a plurality of ports at the second end portion of the nasal prong,
the plurality of ports configured to be fluidically coupled to a pressure sensor such that a pressure measurement associated with the flow passage of the nasal prong can be taken over a time period during the delivery of the flow of the oxygen-containing gas, the pressure measurement associated with a pressure at each port from the plurality of ports.

23. The apparatus of claim 22, wherein the plurality of ports are spaced equidistance from each other around a circumference of the nasal prong.

24. The apparatus of claim 22, wherein the plurality of ports are at an end surface of the second end portion of the nasal prong.

25. The apparatus of claim 22, wherein the plurality of ports are in fluid communication with the flow passage.

26. The apparatus of claim 22, wherein the plurality of ports includes at least one port at an end of the second end portion of the nasal prong and at least one port in fluid communication with the flow passage.

27. The apparatus of claim 22, wherein the pressure measurement is associated with a pressure at each port from the plurality of ports.

28. A method for delivering oxygen-containing gas to an airway of a patient, comprising:
providing a flow of an oxygen-containing gas to a nasal cannula having at least one nasal prong inserted within a nostril of the patient, the nasal prong having a first end portion defining an inlet opening and a second end portion defining an outlet opening and defining a flow passage between the inlet opening and the outlet opening, the nasal prong defining a plurality of ports at the second end portion of the nasal prong, the flow of the oxygen-containing gas having a first flow rate and a first percentage of oxygen within the oxygen-containing gas;
measuring a pressure associated with the flow of the oxygen-containing gas through the flow passage of the nasal prong via a pressure sensor, the measured pressure based on a pressure associated with the flow passage at each port from the plurality of ports.

29. The method of claim 28, wherein the measuring a pressure includes measuring a plurality of pressure measurements during a time period.

30. The method of claim 29, further comprising:
communicating the plurality of pressure measurements to a controller operatively coupled to the pressure sensor and a source of the oxygen-containing gas, the controller including a processor.
determining, at the processor, a pressure waveform characterizing the plurality of pressure measurements as a function of time during the flow of the oxygen-containing gas;
based on the pressure waveform, determining at the processor at least one of an inspiratory flow rate of the patient, a tidal volume of the patient, or a minute ventilation of the patient;
producing, at the processor, a control signal based at least on at least one of the inspiratory flow rate, the tidal volume, or the minute ventilation; and
sending the control signal to the source of the oxygen-containing gas to adjust at least one of a percentage of oxygen within the oxygen-containing gas or a flow rate of the oxygen-containing gas being delivered into the nasal cannula via the flow of the oxygen-containing gas, thereby producing a second flow of the oxygen-containing gas.

31. The method of claim 30, wherein the control signal causes a flow control valve operably coupled to the processor and coupled to the source of the oxygen-containing gas to adjust at least one of the percentage of oxygen within the oxygen-containing gas or the flow rate of the oxygen-containing gas being delivered into the nasal cannula.

32. The method of claim 30, wherein the processor is configured to receive input from a user including data related to a health parameter associated with the patient, and
the control signal produced by the processor is based further on the health parameter associated with the patient.
